(19) United States
(12) Patent Application Publication
(10) Pub. No.: US 2011/0124613 A1
(43) Pub. Date: May 26, 2011

(54) Novel Combination of Compounds to be Used in the Treatment of Airway Diseases, Especially Chronic Obstructive Pulmonary Disease (COPD) and Asthma

(76) Inventors: Tomas Eriksson, Lund (SE); Johan Hansson, Lund (SE); Marguerite Mensonides-Harsema, Hamburg (DE); John Mo, Lund (SE)

(21) Appl. No.: 12/527,754

(22) PCT Filed: Feb. 21, 2008
(86) PCT No.: PCT/SE2008/050204

§ 371 Date: Dec. 8, 2010

Related U.S. Application Data
(60) Provisional application No. 60/891,244, filed on Feb. 23, 2007.

(51) Int. Cl. A61K 31/438; C07D 491/10; C07D 471/10; A61K 31/56; A61P 11/00; A61P 11/06

(52) U.S. Cl. 514/171; 514/278; 546/17

(57) ABSTRACT

The present invention provides a pharmaceutical product comprising, in combination of, (a) a (therapeutically effective) dose of a first active ingredient, which is a compound of formula (I) or a pharmaceutically acceptable salt thereof; and (b) a (therapeutically effective) dose of a second active ingredient, which is a glucocorticoid receptor agonist; and optionally, (c) a (therapeutically effective) dose of a third active ingredient, which is a β₂-agonist. The invention further relates to pharmaceutical compositions comprising said combination and to methods of treating treatment of airway diseases, especially chronic obstructive pulmonary disease (COPD) and asthma in mammals by administrating said combination. The invention further relates to a kit comprising the combination and use of said kit in treatment of airway diseases such as COPD and asthma.

### THE FIELD OF THE INVENTION

The present invention relates to a combination of (a) a chemokine receptor 1 (CCR1) antagonist and (b) a glucocorticoid receptor agonist and optionally (c) a β₂-agonist. The invention further relates to a pharmaceutical composition comprising said combination and to a method of treatment of airway diseases, such as chronic obstructive pulmonary disease (COPD) or asthma in mammals by administrating said combination. The invention further relates to a kit comprising the combination and use of said kit in treatment of airway diseases such as COPD or asthma.

### BACKGROUND OF THE INVENTION

The essential function of the lungs requires a fragile structure with enormous exposure to the environment, including pollutants, microbes, allergens, and carcinogens. Host factors, resulting from interactions of lifestyle choices and genetic composition, influence the response to this exposure. Damage or infection to the lungs can give rise to a wide range of diseases of the respiratory system (or airway diseases). A number of these diseases are of great public health importance. Airway diseases include Acute Lung Injury, Acute Respiratory Distress Syndrome (ARDS), occupational lung disease, lung cancer, tuberculosis, fibrosis, pneumoconiosis, pneumonia, emphysema, Chronic Obstructive Pulmonary Disease (COPD) and asthma.

Among the most common airway diseases is asthma. Asthma is generally defined as an inflammatory disorder of the airways with clinical symptoms arising from intermittent airflow obstruction. It is characterised clinically by paroxysms of wheezing, dyspnea and cough. It is a chronic disabling disorder that appears to be increasing in prevalence and severity. It is estimated that 15% of children and 5% of adults in the population of developed countries suffer from asthma. Therapy should therefore be aimed at controlling symptoms so that normal life is possible and at the same time provide basis for treating the underlying inflammation.

COPD is a term which refers to a large group of lung diseases which can interfere with normal breathing. Current clinical guidelines define COPD as a disease state characterized by airflow limitation that is not fully reversible. The airflow limitation is usually both progressive and associated with an abnormal inflammatory response of the lungs to noxious particles and gases. The most important contributory source of such particles and gases, at least in the western world, is tobacco smoke. COPD patients have a variety of symptoms, including cough, shortness of breath, and excessive production of sputum; such symptoms arise from dysfunction of a number of cellular compartments, including neutrophils, macrophages, and epithelial cells. The two most important conditions covered by COPD are chronic bronchitis and emphysema.

Chronic bronchitis is a long-standing inflammation of the bronchi which causes increased production of mucous and other changes. The patients' symptoms are cough and expectoration of sputum. Chronic bronchitis can lead to more frequent and severe respiratory infections, narrowing and plugging of the bronchi, difficult breathing and disability.

Emphysema is a chronic lung disease which affects the alveoli and/or the ends of the smallest bronchi. The lung loses its elasticity and therefore these areas of the lungs become enlarged. These enlarged areas trap stale air and do not effectively exchange it with fresh air. This results in difficult breathing and may result in insufficient oxygen being delivered to the blood. The predominant symptom in patients with emphysema is shortness of breath.

WO01/98273, WO03/051839 and WO04/005295 describe compounds having activity as pharmaceuticals, in particular as modulators of chemokine receptor (especially MIP-1α chemokine receptor), salts thereof and pharmaceutical compositions, and their potential use in treating various diseases.

The MIP-1α chemokine receptor CCR1 (chemokine receptor 1) is highly expressed in tissues affected in different autoimmune, inflammatory, proliferative, hyperproliferative and immunologically mediated diseases e.g. asthma and chronic obstructive pulmonary disease. Moreover, inflammatory cells (e.g. neutrophils and monocytes/macrophages) contribute to the pathogenesis of airway diseases such as COPD by secretion of proteolytic enzymes, oxidants and pharmacologic mediators. These cells are dependent on the function of CCR1 for recruitment and activation in lung tissues.

Therapeutic agents used in the treatment of airway diseases include glucocorticoid receptor agonists. Glucocorticoid receptor agonists (such as corticosteroids, glucocorticoids and non-steroid glucocorticoid agonists) are potent anti-inflammatory agents. Whilst their exact mechanism of action is not clear, the end result of glucocorticoid receptor agonist treatment is a decrease in the number, activity and movement of inflammatory cells into the bronchial submucosa, leading to decreased airway responsiveness. Glucocorticoid receptor agonists may also cause reduced shedding of bronchial epithelial lining, vascular permeability, and mucus secretion.

Whilst glucocorticosteroid treatment can yield important benefits, the efficacy of these agents may however not always be satisfactory, particularly in COPD. Moreover, whilst the use of steroids may lead to therapeutic effects, it is desirable to be able to use steroids in low doses to minimise the occurrence and severity of undesirable side effects that may be associated with regular administration. Recent studies have also highlighted the problem of the acquisition of steroid resistance amongst patients suffering from airway diseases. For example, cigarette smokers with asthma have been found to be insensitive to short term inhaled corticosteroid therapy, but the disparity of the response between smokers and non-smokers appears to be reduced with high dose inhaled corticosteroid (Tomlinson et al., Thorax 2005;60:282-287).

Therapeutic agents used in the treatment of airway diseases also include beta2 (β₂) adrenoreceptor agonists. These agents (also known as (β₂-agonists) may be used to alleviate symptoms of airway diseases by relaxing the bronchial smooth muscles, reducing airway obstruction, reducing lung hyperinflation and decreasing shortness of breath.

Whilst treatment with a β₂-agonist can yield important benefits, the efficacy of these agents with regard to modifying the disease is not always satisfactory.

Hence there is a pressing medical need for new therapies against airway diseases such as COPD and asthma, in particular for therapies with disease modifying potential.

WO2004005295, WO2005049620, WO2005061499 describe compounds having activity as pharmaceuticals, in particular as modulators of chemokine receptor (especially MIP-1α chemokine receptor), salts thereof and pharmaceutical formulations, and their potential use in treating various diseases.

The present invention relates to a combination of a CCR1 antagonist with a glucocorticoid receptor agonist and optionally a β₂-agonist.

It is contemplated that the combination of the present invention has a beneficial therapeutic effect in the treatment of airway diseases. For example, the combination according to the invention is considered to be particularly effective in reducing inflammatory cell influx into the lung. The beneficial effect may be observed when the two (or three) active substances are administered simultaneously (either in a single pharmaceutical composition or in separate compositions), or sequentially or separately.

### DETAILED DESCRIPTION OF THE INVENTION

Thus, according to the present invention, there is provided a pharmaceutical product comprising, in combination,

(a) a first active ingredient, which is a compound of general formula (I):

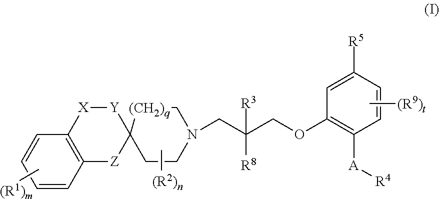

wherein:

m is 0, 1 or 2;

R¹ is halogen, cyano or C₁₋₆haloalkyl;

X, Y and Z are independently a bond, —O—, —NH—, CH₂— or —C(O)—, provided that only one of X, Y and Z is a bond, and provided that X and Y are not simultaneously —O— or —C(O)—;

n is 0, 1 or 2;

R² is ═O or C₁₋₆alkyl;

q is 0 or 1;

R³ is hydrogen, hydroxyl or NH₂;

R⁸ is hydrogen or C₁₋₆alkyl;

A is a bond or C₁₋₃alkyl;

R⁴ is hydrogen, hydroxyl, oxo, NHC(O)R¹⁰, C(O)NR¹¹R¹², COOR¹³ or SO₃R¹³;

R⁵ is hydrogen, halogen, hydroxyl or C₁₋₆alkoxy, optionally substituted by one or more substituent independently selected from halogen, cyano, hydroxyl and carboxyl;

t is 0, 1 or 2;

R⁹ is halogen, cyano, C₁₋₃alkoxy or C₁₋₃haloalkyl;

R¹⁰ is hydrogen, C₁₋₃alkyl, NR¹¹R¹² or OR¹³;

R¹¹ and R¹² are independently selected from hydrogen, C₁₋₆alkyl and C₃₋₇cycloalkyl, or R¹¹ and R¹² together with the nitrogen atom to which they are attached form a 4 to 7-membered heterocyclic ring, which may optionally be substituted by one or more hydroxyl groups; and

R¹³ is hydrogen or C₁₋₃alkyl,

or a pharmaceutically acceptable salt thereof; and

(b) a second active ingredient, which is a glucocorticoid receptor agonist; and optionally

(c) a third active ingredient, which is a I3₂-agonist, provided the agonist is not selected from a N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide or a salt thereof,

a N-[2-(Diethylamino)ethyl]-N-(2-{[2-(4-hydroxy-2-oxo-2,3-dihydro-1,3-benzothiazol-7-yl)ethyl]amino}ethyl)-3-[2-(3-chlorophenyl)ethoxy]propanamide or a salt thereof, or

a 7-[(1R)-2-({2-[(3-{[2-(2-Chlorophenyl)ethyl]amino}propyl)-thio]ethyl}amino-1-hydroxyethyl]-4-hydroxy-1,3-benzothiazol-2(3H)-one or a salt thereof.

One embodiment relates to provided a pharmaceutical product comprising, in combination,

(a) a first active ingredient, which is a compound of general formula (I) wherein:

m is 1;

R¹ is halogen; X, Y and Z are independently a bond, —O— or CH₂—, provided that only one of X, Y and Z is a bond; n is 0; q is 1; R³ is hydroxyl; R⁸ is hydrogen; A is a bond; R⁴ is C(O)NR¹¹ R¹²; R⁵ is C₁₋₆alkoxy, optionally substituted by one or more substituent independently selected from hydroxyl and carboxyl; t is 1; R⁹ is halogen; R¹¹ and R¹² are independently selected from hydrogen and C₁₋₆alkyl; or a pharmaceutically acceptable salt thereof; and

(b) a second active ingredient, which is a glucocorticoid receptor agonist; and optionally

(c) a third active ingredient, which is a β₂-agonist, provided the agonist is not selected from

a N-[2-(Diethylamino)ethyl]-N-(2-{[2-(4-hydroxy-2-oxo-2,3-dihydro-1,3-benzothiazol-7-yl)ethyl]amino)ethyl}-3-[2-(1-naphthyl)ethoxy]propanamide or a salt thereof,

a N-[2-(Diethylamino)ethyl]-N-(2-{[2-(4-hydroxy-2-oxo-2,3-dihydro-1,3-benzothiazol-7-yl)ethyl]amino}ethyl)-3-[2-(3-chlorophenyl)ethoxy]propanamide or a salt thereof, or

a 7-[(1R)-2-({2-[(3-{[2-(2-Chlorophenyl)ethyl]amino}propyl)-thio]ethyl}amino)-1-hydroxyethyl]-4-hydroxy-1,3-benzothiazol-2(3H)-one or a salt thereof.

In another embodiment the glucocorticoid receptor agonist is budesonide.

In yet a further embodiment I3₂-agonist is selected from any of formoterol, indacaterol or selected from

a N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide or a salt thereof,

a N-[2-(Diethylamino)ethyl]-N-(2-{[2-(4-hydroxy-2-oxo-2,3-dihydro-1,3-benzothiazol-7-yl)ethyl]amino}ethyl)-3-[2-(3-chlorophenyl)ethoxy]propanamide or a salt thereof, or a 7-[(1R)-2-({2-[(3-{[2-(2-Chlorophenyl)ethyl]amino}propyl)-thio]ethyl}amino-1-hydroxyethyl]-4-hydroxy-1,3-benzothiazol-2(3H)-one or a salt thereof.

In one embodiment R¹ is selected from chlorine and fluorine. In one embodiment R¹ is chlorine.

In one embodiment X is —O—, Y is a bond and Z is CH₂. In yet another embodiment X is a bond, Y is —NH—, and Z is —C(O). In yet another embodiment, X is —CH₂, Y is —O— and Z is a bond.

In one embodiment q is 1.

In one embodiment n is 0. In another embodiment n is 1 or 2.

In yet another embodiment R³ is hydrogen, hydroxyl or amino group. In one embodiment R³ is hydrogen. In yet another embodiment R³ is hydroxyl. In one embodiment R³ is an NH₂.

In yet a further embodiment of the present invention, R⁸ is hydrogen, methyl, ethyl, n-propyl, isopropyl, n-butyl, isobutyl or tert-butyl. In one embodiment, R⁸ is hydrogen. In one embodiment, R⁸ is methyl.

In yet another embodiment R³ is hydroxyl and R⁸ is hydrogen.

In another embodiment m is 1, R¹ is chloride, n is 0, p is 1, R⁸ is hydrogen and R³ is hydroxyl.

In one embodiment R⁴ is —CONR¹¹R¹² and suitable groups R¹¹ and R¹² are selected from hydrogen, methyl, ethyl, n-propyl, isopropyl, n-butyl, isobutyl or tert-butyl. In one embodiment, R¹¹ is hydrogen and R¹² is methyl. In another embodiment R¹¹ and R¹² are both methyl.

In another embodiment, R¹¹ and R¹² together with the nitrogen atom to which they are attached form a 4 to 7-membered heterocyclic ring, which is optionally substituted with one or more hydroxy groups. In one embodiment heterocyclic groups for R¹¹ and R¹² and the nitrogen atom to which they are attached include azetidinyl, pyrrolidinyl, piperidinyl and pyrrolidinyl.

In one embodiment R⁴ is —N(H)C(O)NR¹¹R¹² wherein R¹¹ and R¹² are hydrogen, methyl, ethyl, n-propyl, isopropyl, n-butyl, isobutyl or tert-butyl. In one embodiment, R¹¹ is hydrogen and R¹² is methyl. In another embodiment R¹¹ and R¹² are both methyl.

In yet another embodiment A is a bond. In one embodiment A is methyl or an ethyl linker

In one embodiment R⁵ is hydrogen, halogen, hydroxyl, methoxy, ethoxy, n-propoxy, isopropoxy, n-butoxy, isobutoxy or tert-butoxy, optionally substituted with halogen, hydroxyl or carboxyl. In one embodiment R⁵ is hydrogen. In another embodiment R⁵ is halogen such as fluorine. In another embodiment R⁵ is hydroxyl. In yet another embodiment R⁵ is —OCH₂COOH. In yet a further embodiment R⁵ is —OC(CH₃)₂COOH.

In another embodiment of the present invention R⁵ is selected from —OCH₂CF₃, —OCH₂CH₂CF₃, —OCH₂CHF₂ or —OCH₂CN.

In a further embodiment of the present invention R⁹ is a halogen, such as chlorine and fluorine. In one embodiment t is 1 and R⁹ is chlorine.

In another embodiment R¹⁰ is methyl.

In one embodiment R⁴ represents a group —CONR¹¹R¹², R¹¹ is hydrogen and R¹² is methyl, A is a bond, R⁵ is —OC(CH₃)₂COOH, t is 1 and R⁹ is chlorine.

For the avoidance of doubt, the present invention relates to a pharmaceutical product whereby the glucocorticoid receptor agonist (and optionally the (β₂-agonist) is combined with any compound falling within the scope of compounds of formula (I) as defined above.

For the avoidance of doubt it is to be understood that where in this specification a group is qualified by ‘hereinbefore defined’, ‘defined hereinbefore’ or ‘defined above’ the said group encompasses the first occurring and broadest definition as well as each and all of the other definitions for that group.

For the avoidance of doubt it is to be understood that in this specification ‘C₁₋₆’ means a carbon group having 1, 2, 3, 4, 5 or 6 carbon atoms.

In this specification, unless stated otherwise, the term “alkyl” includes both straight and branched chain alkyl groups and may be, but are not limited to methyl, ethyl, n-propyl, i-propyl, n-butyl, i-butyl, s-butyl, t-butyl, n-pentyl, i-pentyl, neo-pentyl, n-hexyl or i-hexyl. The term C₁₋₄ alkyl having 1 to 4 carbon atoms and may be but are not limited to methyl, ethyl, n-propyl, i-propyl or tent-butyl.

The term “alkoxy”, unless stated otherwise, refers to radicals of the general formula —O—R, wherein R is selected from a hydrocarbon radical. The term “C₁₋₆alkoxy” may include, but is not limited to methoxy, ethoxy, propoxy, isopropoxy, butoxy, t-butoxy, isobutoxy, cyclopropylmethoxy, allyloxy or propargyloxy.

The term ‘C₁₋₆alkoxy substituted with carbonyl and hydroxyul, includes for example substituent —OC(CH₃)₂COOH.

In this specification, unless stated otherwise, the term “cycloalkyl” refers to an optionally substituted, partially or completely saturated monocyclic, bicyclic or bridged hydrocarbon ring system. The term “C₁₋₆cycloalkyl” may be, but is not limited to cyclopropyl, cyclobutyl, cyclopentyl or cyclohexyl.

In this specification, unless stated otherwise, the term “4 to 7-membered heterocyclic ring” refers to a ringsystem having, in addition to carbon atoms, zero to three heteroatoms, including the oxidized form of nitrogen and sulfur and any quaternized form of a basic nitrogen, including, but not limited to cyclopropane, oxirane, cyclobutane, azetidine, cyclopentane, cyclohexane, benzyl, furane, thiophene, pyrrolidine, morpholine, piperidine, piperazine, pyrazine, azepane.

In this specification, unless stated otherwise, the terms “halo” and “halogen” may be fluorine, iodine, chlorine or bromine.

In this specification, unless stated otherwise, the term “haloalkyl” means an alkyl group as defined above, which is substituted with halogen as defined above. The term “C₁-C₆haloalkyl” may include, but is not limited to fluoromethyl, difluoromethyl, trifluoromethyl, fluoroethyl, difluoroethyl or bromopropyl. The term “C₁₋₃haloalkylO” may include, but is not limited to fluoromethoxy, difluoromethoxy, trifluoromethoxy, fluoroethoxy or difluoroethoxy. The term “halophenyl” may include, but is not limited to fluorophenyl, difluorophenyl, trifluorophenyl, chlorophenyl, dichlorophenyl or trichlorophenyl.

It will be appreciated that throughout the specification, the number and nature of substituents on rings in the compounds of the invention will be selected so as to avoid sterically undesirable combinations.

In another embodiment of the present invention, the compound of formula (I) is selected from: N-(2-{[(2S)-3-(5-chloro-1′H,3H-spiro[1-benzofuran-2,4′-piperidin]-1′-yl)-2-hydroxypropyl]oxy}-4-hydroxyphenyl)acetamide;

N-(2-{[(2S)-3-(5-chloro-1′H-spiro[1,3-benzodioxole-2,4′-piperidin]-1′-yl)-2-hydroxypropyl]oxy}-4-hydroxyphenyl)acetamide trifluoroacetate (salt);

2-{[(2S)-3-(5-chloro-1′H,3H-spiro[1-benzofuran-2,4′-piperidin]-1′-yl)-2-hydroxypropyl]oxy}-4-hydroxy-N-methylbenzamide trifluoroacetate (salt);

2-{[(2S)-3-(5-chloro-1′H,3H-spiro[1-benzofuran-2,4′-piperidin]-1′-yl)-2-hydroxypropyl]oxy}-4-hydroxybenzoic acid trifluoroacetate (salt);

N-(2-{[(2S)-3-(5-chloro-1′H,3H-spiro[2-benzofuran-1,4′-piperidin]-1′-yl)-2-hydroxypropyl]oxy}-4-hydroxyphenyl)acetamide trifluoroacetate (salt);

2-{[(2S)-3-(5-chloro-1′H,3H-spiro[2-benzofuran-1,4′-piperidin]-1′-yl)-2-hydroxypropyl]oxy}-4-hydroxy-N-methylbenzamide;

N-(2-{[(2S)-3-(5-fluoro-1′H,3H-spiro[1-benzofuran-2,4′-piperidin]-1′-yl)-2-hydroxypropyl]oxy}-4-hydroxyphenyl)acetamide;

2-{[(2S)-3-(5-fluoro-1′H,3H-spiro[1-benzofuran-2,4′-piperidin]-1′-yl)-2-hydroxypropyl]oxy}-4-hydroxy-N-methylbenzamide;

N-[2-({(2S)-3-[(2R)-5-chloro-1′H,3H-spiro[1-benzofuran-2,3′-pyrrolidin]-1′-yl]-2-hydroxypropyl}oxy)-4-hydroxyphenyl]acetamide;

N-(2-{[(2S)-3-(5-chloro-1′H,3H-spiro[1-benzofuran-2,4′-piperidin]-1′yl)-2-hydroxypropyl]oxy}-4-hydroxyphenyl)urea trifluoroacetate (salt);

4-fluoro-2-{[(2S)-3-(5-fluoro-1′H,3H-spiro[1-benzofuran-2,4′-piperidin]-1′-yl)-2-hydroxypropyl]oxy}benzoic acid hydrochloride;

N-(2-{[(2S)-3-(5-chloro-1′H,3H-spiro[1-benzofuran-2,4′-piperidin]-1′-yl)-2-hydroxypropyl]oxy}-4-fluorophenyl)urea trifluoroacetate (salt);

N-(2-{[(2S)-2-amino-3-(5-fluoro-1′H,3H-spiro[1-benzofuran-2,4′- piperidin]-1′-yl)propyl]oxy}-4-hydroxyphenyl)acetamide bis(trifluoroacetate) (salt);

Benzaldehyde, 2-[(2S)-3-(5-chlorospiro[benzofuran-2(3H),4′-piperidin]-1′-yl)-2-hydroxypropoxy]- ;

Spiro[benzofuran-2(3H),4′-piperidine]-1′-ethanol, 5-chloro-α-[[2-(2-hydroxyethyl)phenoxy]methyl]-, (αS)—;

Spiro[benzofuran-2(3H),4′-piperidine]-1′-ethanol, 5-chloro-α-[[2-(hydroxymethyl)phenoxy]methyl]-, (αS)—;

N-(2-{[(2S)-3-(5-chloro-1′H,3H-spiro[1-benzofuran-2,4′-piperidin]-1′-yl)-2-hydroxypropyl]oxy}-5-chloro-4-hydroxyphenyl)acetamide;

2-Chloro-5-{[(2S)-3-(5-chloro-1′H,3H-spiro[1-benzofuran-2,4′-piperidin]-1′-yl)-2-hydroxypropyl]oxy}-(4-{acetylamino}phenoxy)acetic acid;

5-{[(2S)-3-(5-Chloro-1′H,3H-spiro[1-benzofuran-2,4′-piperidin]-1′-yl)-2-hydroxypropyl]oxy}-(4-{acetylamino}phenoxy)acetic acid;

{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}acetic acid;

2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid;

(2-Chloro-5-{[(2S)-3-(5-chloro-1′H,3H-spiro[1-benzofuran-2,4′-piperidin]-1′-yl)-2-hydroxypropyl]oxy}-4-{[(3S)-3-hydroxypyrrolidin-1-yl]carbonyl}phenoxy)acetic acid;

5-Chloro-2-{[(2S)-3-(5-chloro-1′H,3H-spiro[1-benzofuran-2,4′-piperidin]-1′-yl)-2-hydroxypropyl]oxy}-4-(cyanomethoxy)benzoic acid tifluoroacetate (salt);

2-{[(2S)-3-(5-chloro-1′H,3H-spiro[1-benzofuran-2,4′-piperidin]-1′-yl)-2-hydroxypropyl]oxy}-5-chloro-4-(2,2-difluoroethoxy)benzoic acid trifluoroacetate (salt);

5-Chloro-2-{[(2S)-3-(5-chloro-1′H,3H-spiro[1-benzofuran-2,4′-piperidin]-1′-yl)-2-hydroxypropyl]oxy}-4-(3,3,3-trifluoropropoxy)benzoic acid trifluoroacetate (salt);

N-(2-{3-[5-chloro-1′H,3H-spiro[1-benzofuran-2,4′-piperidin]-1′-yl]propoxy}phenyl)acetamide trifluoroacetate (salt);

Methyl 3-(2-{[(2S)-3-(5-chloro-1′H3H-spiro[1-benzofuran-2,4′-piperidin]-1′-yl)-2-hydroxypropyl]oxy}-4-fluorophenyl)propanoic acid trifluo acetic acid salt;

N-(2-{[(2S)-3-({spiro[indole-2-4′-piperidin]-3(1H)-one}-1′-yl)-2-hydroxypropyl]oxy}-4-hydroxyphenyl)acetamide; and

(2-{[(2S)-3-(5-Chloro-1′H,3H-spiro[1-benzofuran-2,4′-piperidin]-1′-yl)-2-hydroxypropyl]oxy}-4-fluorophenyl)methanesulfonic acid

or a pharmaceutically acceptable salt, solvate or solvated salt thereof

For the avoidance of doubt, the present invention relates to a pharmaceutical product whereby the glucocorticoid receptor agonist (and optionally the β₂-agonist) is combined with any one of the specific compounds of formula (I) mentioned above.

The compounds of formula (I) according to the present invention may be synthesised using the procedures set out in WO2004/005295, WO2005049620, WO2005061499 and WO2008/010765.

In one embodiment of the present invention relates to a pharmaceutical product comprising, in combination,

(a) a first active ingredient, which is 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid or pharmaceutically acceptable salts, solvate or solvated salt thereof; and

(b) a second active ingredient, which is a glucocorticoid receptor agonist; and optionally

(c) a third active ingredient, which is a β₂-agonist, provided the agonist is not selected from a N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide or a salt thereof,

a N-[2-(Diethylamino)ethyl]-N-(2-{[2-(4-hydroxy-2-oxo-2,3-dihydro-1,3-benzothiazol-7-yl)ethyl]amino}ethyl)-3-[2-(3-chlorophenyl)ethoxy]propanamide or a salt thereof, or

a 7-[(1R)-2-({2-[(3-{[2-(2-Chlorophenyl)ethyl]amino}propyl)-thio]ethyl}amino)-1-hydroxyethyl]-4-hydroxy-1,3-benzothiazol-2(3H)-one or a salt thereof.

In another embodiment the glucocorticoid receptor agonist is budesonide.

In yet a further embodiment β₂-agonist is selected from any of formoterol, indacaterol or a N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide or a salt thereof,

a N-[2-(Diethylamino)ethyl]-N-(2-{[2-(4-hydroxy-2-oxo-2,3-dihydro-1,3-benzothiazol-7-yl)ethyl]amino}ethyl)-3-[2-(3-chlorophenyl)ethoxy]propanamide or a salt thereof, or

a 7-[(1R)-2-({2-[(3-{[2-(2-Chlorophenyl)ethyl]amino}propyl)-thio]ethyl}amino)-1-hydroxyethyl]-4-hydroxy-1,3-benzothiazol-2(3H)-one or a salt thereof.

The preparation of 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid is described in WO2008/010765.

The compounds of formula (I) are capable of existing in stereoisomeric forms. It will be understood that the invention encompasses the use of all geometric and optical isomers of the compounds of formula (I) and mixtures thereof including racemates. The use of tautomers and mixtures thereof also form an aspect of the present invention. Preferred optical isomers are the (S)-enantiomers (i.e. compounds with the S configuration at the stereocentre with R⁸ and OH attached).

The compounds of formula (I) may be used in the form of a pharmaceutically acceptable salt thereof, conceivably an acid addition salt such as a hydrochloride, hydrobromide, phosphate, sulfphate, acetate, ascorbate, benzoate, fumarate, furoate, succinate, maleate, tartrate, citrate, oxalate, xinafoate, methanesulphonate, p-toluenesulphonate, trifluoroacetate, sodium, hemifumarate, 2-fluorobenzoate or 2,6-difluorobenzoate. Pharmaceutically acceptable salts may also be formed together with metals such as calcium, magnesium, sodium, potassium or zinc or bases such as piperazine, 2-aminoethanol, choline, diethylamine or diethanol amine. Furthermore, a compound of formula (I) may be used in the form of a pharmaceutically acceptable salt thereof, like an amino acid addition salt such as L-lysine, glycine, L-glutamine, L-asparagine or L-arganine. A pharmaceutically acceptable salt also includes an internal salt (zwitterionic) form. Any reference to compounds of formula (I) or salts thereof also encompasses solvates of such compounds and solvates of such salts (e.g. hydrates).

In yet a further embodiment of the present invention, the compound of formula (I) is a hydrochloride, trifluoroacetate, p-toluensulfonate, sodium, hemifumarate, furoate, benzoate, 2-fluorobenzoate or 2,6-difluorobenzoate salt of 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid as described in WO2008/010765.

It will be appreciated that the compounds of formula (I) and salts thereof may exist as zwitterions. Thus, whilst the compounds are drawn and referred to in the neutral form, they may exist also in internal salt (zwitterionic) form. The representation of formula (I) and the examples of the present invention covers both neutral and zwitterionic forms and mixtures thereof in all proportions.

In one embodiment of the invention a compound of formula (I) is 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid, which exhibits at least the following characteristic X-ray powder diffraction peaks (expressed in degrees 2θ) (Form A):

(1) 5.1, 10.2 and 12.9, or

(2) 5.1, 8.9 and 13.2, or

(3) 8.9, 10.2, 12.9, 15.1, 17.0 and 21.2 or

(4) 5.1, 8.9, 10.2, 14.6, 15.4, 21.2 and 25.8 or

(5) 5.1, 8.9, 10.2, 12.6, 14.6, 15.1 and 17.0 or

(6) 5.1, 10.2, 12.6, 13.2, 14.6, 15.1, 17.0, 17.9, 21.2 and 21.8 or

(7) 5.1, 8.9, 10.2, 12.6, 13.2, 14.6, 14.9, 16.4, 19.2, 21.8 and 27.1 or

(8) 5.1, 8.9, 10.2, 12.6, 12.9, 13.2, 14.6, 14.9, 15.1, 15.4, 16.4, 17.9, 19.2, 20.0, 21.8 and 25.8.

In another embodiment of the invention a compound of formula (I) is 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid, which exhibits at least the following characteristic X-ray powder diffraction peaks (expressed in degrees 2θ) (Form C):

(1) 4.5, 8.9 and 12.8, or

(2) 4.5, 8.6 and 10.6, or

(3) 4.5, 8.9, 10.6, 12.8, 14.8 and 17.6 or

(4) 8.6, 8.9, 12.8, 13.9, 15.7, 16.6 and 18.8 or

(5) 4.5, 8.6, 8.9, 10.6, 13.9, 15.7, 16.0, 16.6 and 17.9 or

(6) 4.5, 8.9, 10.6, 12.8, 13.9, 14.8, 15.7, 17.6, 18.8 and 20.0 or

(7) 4.5, 8.6, 8.9, 10.6, 12.8, 13.9, 15.7, 16.0, 16.6, 17.9, 18.8, 20.0, 20.9 and 21.2.

The preparation of polymorphs Form A and C is described in WO2008/010765.

For the avoidance of doubt, the present invention relates to a pharmaceutical product whereby a glucocorticoid receptor agonist (and optionally a β₂-agonist) is combined with any one of the salts or polymorphic forms of a compound of formula (I) mentioned above.

One embodiment of the invention refers to a novel compound selected from 5-Chloro-2-{[(2S)-3-(5-chloro-1′H,3H-spiro[1-benzofuran-2,4′-piperidin]-1′-yl)-2-hydroxypropyl]oxy}-4-(cyanomethoxy)benzoic acid;

2-{[(2S)-3-(5-chloro-1′H,3H-spiro[1-benzofuran-2,4′-piperidin]-1′-yl)-2-hydroxypropyl]oxy}-5-chloro-4-(2,2-difluoroethoxy)benzoic acid;

5-Chloro-2-{[(2S)-3-(5-chloro-1′H,3H-spiro[1-benzofuran-2,4′-piperidin]-1′-yl)-2-hydroxypropyl]oxy}-4-(3,3,3-trifluoropropoxy)benzoic acid;

N-(2-{3-[5-chloro-1′H,3H-spiro[1-benzofuran-2,4′-piperidin]-1′-yl]propoxy}phenyl)acetamide;

Methyl 3-(2-{[(2S)-3-(5-chloro-1′H3H-spiro[1-benzofuran-2,4′-piperidin]-1′-yl)-2-hydroxypropyl]oxy}-4-fluorophenyl)propanoic acid trifluo acetic; and

N-(2-{[(2S)-3-({spiro[indole-2-4′-piperidin]-3(1H)-one}-1′-yl)-2-hydroxyphenyl)acetamide,

or a pharmaceutically acceptable salt, solvate or solvated salt thereof.

In a further embodiment the present invention provides each individual product of the examples presented below.

Process for the Preparation of a Compound of Formula (I)

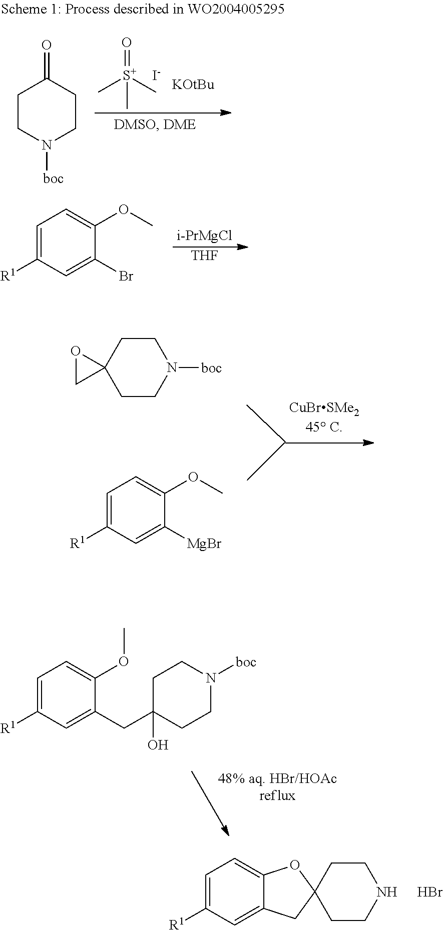

Scheme 2: Starting Phenol Described in WO200012468

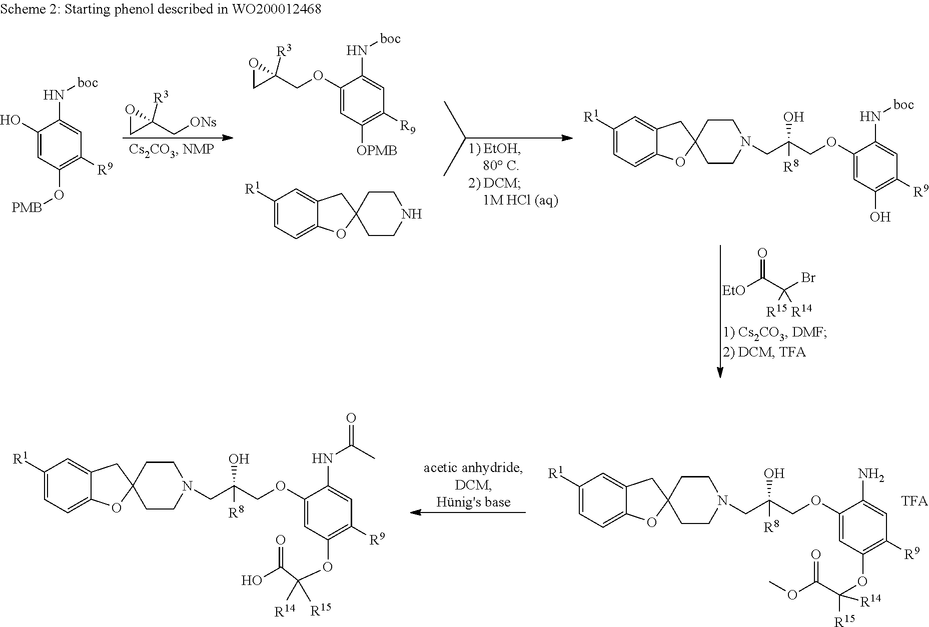

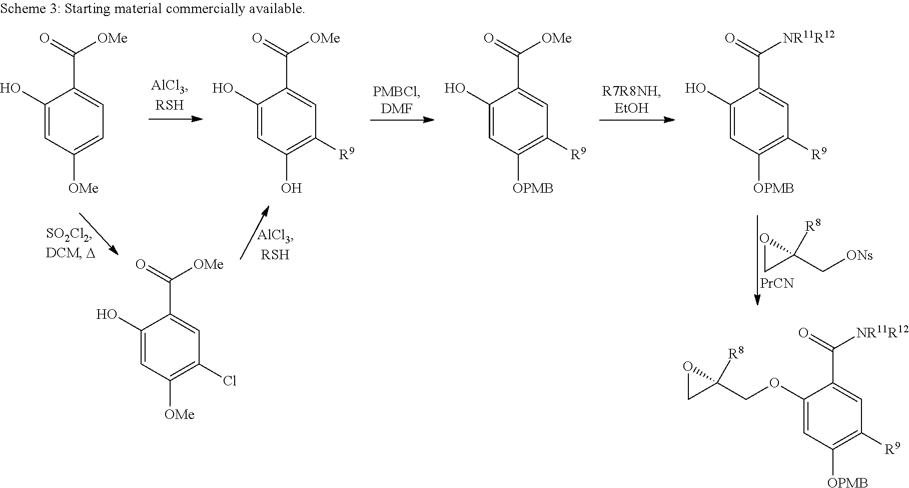

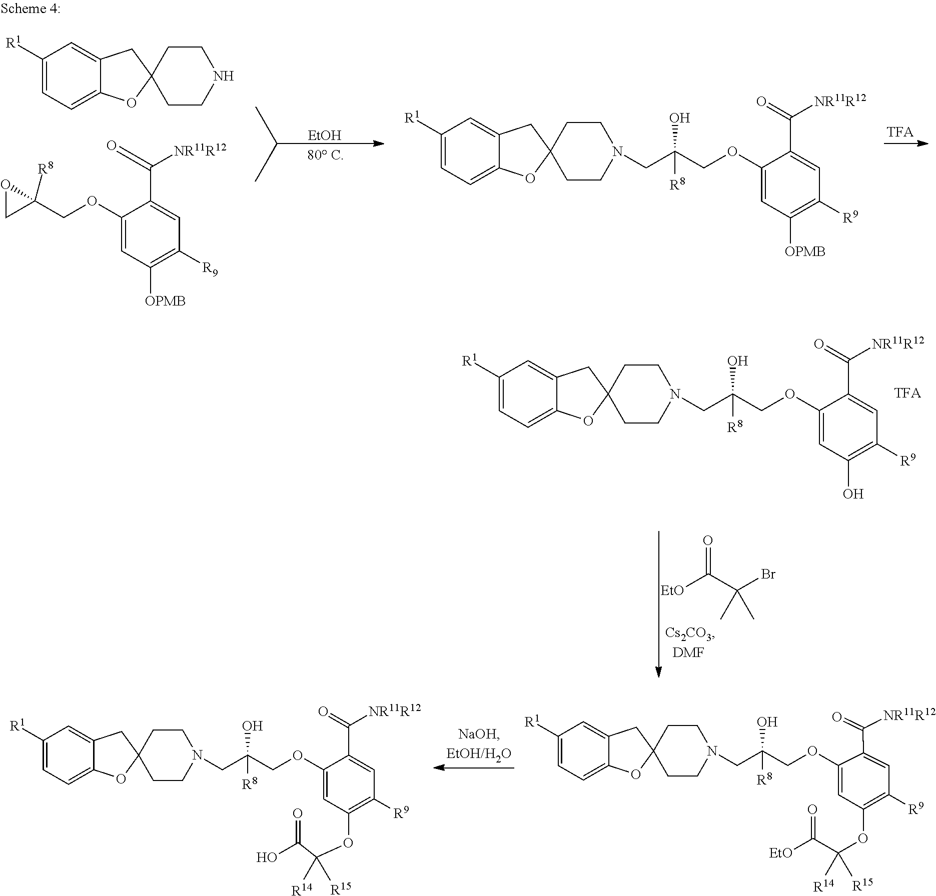

The second active ingredient in the combination of the present invention is a glucocorticoid receptor agonist. The glucocorticoid receptor agonist of the present invention may be any synthetic or naturally occurring glucocorticoid receptor agonist. Examples of glucocorticoid receptor agonist that may be used in accordance with the present invention include budesonide, fluticasone (e.g. as propionate ester), mometasone (e.g. as furoate ester), beclomethasone (e.g. as 17-propionate or 17,21-dipropionate esters), ciclesonide, loteprednol (as e.g. etabonate), etiprednol (e.g. as dicloacetate), triamcinolone (e.g. as acetonide), flunisolide, zoticasone, flumoxonide, rofleponide, butixocort (e.g. as propionate ester), prednisolone, prednisone, tipredane, steroid esters described in WO 2002/12265, WO 2002/12266 and WO 2002/88167 e.g. 6α,9α-difluoro-17α-[(2-furanylcarbonyl)oxy]-11β-hydroxy-16α-methyl-3-oxo-androsta-1,4-diene-17β-carbothioic acid S-fluoromethyl ester, 6α,9α-difluoro-11β-hydroxy-16α-methyl-3-oxo-17α-propionyloxy-androsta-1,4-diene-17β-carbothioic acid S-(2-oxo-tetrahydro-furan-3S-yl)ester and 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester, steroid esters such as those described in DE 4129535, steroids e.g. GSK870086, GSK685698, GSK799943 and those described in WO 2002/00679, WO 2005/041980, and the like.

In the context of the present specification, unless otherwise indicated any reference to a glucocorticoid receptor agonist includes all active salts, solvates, cocrystals or derivatives that may be formed from said glucocorticoid receptor agonist. Examples of possible salts or derivatives of glucocorticoid receptor agonist s include; sodium salts, sulphobenzoates, phosphates, isonicotinates, acetates, propionates, dihydrogen phosphates, palmitates, pivalates, fumarates and pharmaceutically acceptable esters (e.g. C₁-C₆ alkyl esters). Glucocorticoid receptor agonist s and active salts or derivatives thereof may also be in the form of their solvates, e.g. hydrates as well as in the form of cocrystals.

In a further embodiment of the invention a glucocorticoid receptor agonist is budesonide, fluticasone (e.g. as propionate ester), mometasone (e.g. as furoate ester), beclomethasone (e.g. as 17-propionate or 17,21-dipropionate esters), ciclesonide, loteprednol (as e.g. etabonate), etiprednol (e.g. as dicloacetate), triamcinolone (e.g. as acetonide), flunisolide, zoticasone, flumoxonide, rofleponide, butixocort (e.g. as propionate ester), prednisolone, prednisone, tipredane, 6α,9α-difluoro-17α-[(2-furanylcarbonyl)oxy]-11β-hydroxy-16α-methyl-3-oxo-androsta-1,4-diene-17β-carbothioic acid S-fluoromethyl ester, 6α,9α-difluoro-11β-hydroxy-16α-methyl-3-oxo-17α-propionyloxy-androsta-1,4-diene-17β-carbothioic acid S-(2-oxo-tetrahydro-furan-3S-yl) ester, or 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester.

For the avoidance of doubt, the present invention relates to a pharmaceutical product whereby any one of the specific glucocorticoid receptor agonists mentioned above is combined with a compound of formula (I), as defined above (i.e. any one of the compounds falling within the scope of formula (I) as defined above or any one of the specific compounds or salts or polymorphic forms of compounds of formula (I) mentioned above) or a pharmaceutically acceptable salt thereof (and optionally a β₂-agonist).

In one embodiment of the present invention the glucocorticoid receptor agonist is budesonide. The chemical name for budesonide is 16,17-[butylidenebis(oxy)]-11,21-dihydroxy-pregna-1,4-diene-3,20-dione). Budesonide and its preparation is described, for example, in Arzneimittel-Forschung (1979), 29 (11), 1687-1690, DE 2,323,215 and U.S. Pat. No. 3,929,768. Presently available formulations of budesonide are marketed under the tradename ‘Entocort’.

β₂-agonists may be used to alleviate symptoms of airway diseases by relaxing the bronchial smooth muscles, reducing airway obstruction, reducing lung hyperinflation and decreasing shortness of breath. The β₂-agonist of the present invention may be any compound or substance capable of stimulating the β₂-receptor and acting as a β₂-agonist. Examples of β₂-agonists that may be used in the present invention include bambuterol, bitolterol, carbuterol, indacaterol, clenbuterol, fenoterol, formoterol, hexoprenaline, ibuterol, pirbuterol, procaterol, reproterol, salmeterol, sulphonterol, terbutaline, tolubuterol, TA 2005 (chemically identified as 2(1H)-quinolone, 8-hydroxy-5-[1-hydroxy-2-[[2-(4-methoxy-phenyl)-1-methylethyl]-amino]ethyl]-monohydrochloride, [R—(R*,R*)] also identified by Chemical Abstract Service Registry Number 137888-11-0 and disclosed in U.S. Pat. No 4,579,854 (=CHF-4226), GSK159797, formanilide derivatives e.g. 3-(4-{[6-({(2R)-2-[3-(formylamino)-4-hydroxyphenyl]-2-hydroxyethyl}amino)hexyl]oxy}-butyl)-benzenesulfonamide as disclosed in WO 2002/76933, benzenesulfonamide derivatives e.g. 3-(4-{[6-({(2R)-2-hydroxy-2-[4-hydroxy-3-(hydroxy-methyl)phenyl]ethyl{amino)-hexyl]oxy}butyl)benzenesulfonamide as disclosed in WO 2002/88167, aryl aniline receptor agonists such as disclosed in WO 2003/042164 and WO 2005/025555, and indole derivatives such as disclosed in WO 2004/032921; or the gaonist is selected from a N42-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide or a salt thereof,

a N-[2-(Diethylamino)ethyl]-N-(2-{[2-(4-hydroxy-2-oxo-2,3-dihydro-1,3-benzothiazol-7-yl)ethyl]amino}ethyl)-3-[2-(3-chlorophenyl)ethoxy]propanamide or a salt thereof, or a 7-[(1R)-2-({2-[(3-{[2-(2-Chlorophenyl)ethyl]amino}propyl)-thio]ethyl}amino)-1-hydroxyethyl]-4-hydroxy-1,3-benzothiazol-2(3H)-one or a salt thereof.

In one embodiment the β₂-agonist is a long acting β₂-agonist, i.e. a β₂-agonist with activity that persists for more than 12 hours. Examples of long acting β₂-agonists include formoterol, bambuterol and salmeterol.

In one embodiment the β₂-agonist of the invention has a fast onset of action, i.e. a β₂-agonist with an onset of action within 1 hour. Examples of β₂-agonists with fast onset of action include formoterol, TA 2005, salbutamol and β₂-agonists as disclosed in WO2005095328 and US2005272769.

In the context of the present specification, unless otherwise stated, any reference to a β₂-agonists includes active salts, solvates or derivatives that may be formed from said β₂-agonist and any enantiomers and mixtures thereof, including racemates. Examples of possible salts or derivatives are acid addition salts such as the salts of hydrochloric acid, hydrobromic acid, sulphuric acid, phosphoric acid, methanesulphonic acid, acetic acid, fumaric acid, succinic acid, lactic acid, citric acid, tartaric acid, 1-hydroxy-2-naphthalenecarboxylic acid, maleic acid, and pharmaceutically acceptable esters (e.g. C₁-C₆ alkyl esters). The β₂-agonists (including salts and derivatives thereof) may also be in the form of a solvate, e.g. a hydrate.

In an embodiment of the present invention the β₂-agonist is formoterol. The chemical name for formoterol is N-[2-hydroxy-5-[(1)-1-hydroxy-2-[[(1)-2-(4-methoxyphenyl)-1-methylethyl]amino]ethyl]phenyl]-formamide. The preparation of formoterol is described, for example, in WO 92/05147. As will be clear from the above, the term formoterol is intended to include all pharmaceutically acceptable salts thereof In one aspect of this embodiment, the β₂-agonist is formoterol fumarate, for example formoterol fumarate dihydrate.

As emphasised above, it will be understood that the invention encompasses the use of all optical isomers of formoterol and mixtures thereof including racemates. Thus for example, the term formoterol encompasses N-[2-hydroxy-5-[(1R)-1-hydroxy-2-[[(1R)-2-(4-methoxyphenyl)-1-methylethyl]amino]ethyl]phenyl]-formamide, N-[2-hydroxy-5-[(1S)-1-hydroxy-2-[[(1S)-2-(4-methoxyphenyl)-1-methylethyl]amino]ethyl]phenyl]-formamide or a mixture of such enantiomers, including a racemate.

In a further embodiment of the present invention, the β₂-agonist is indacaterol. As will be clear from the above, the term indacaterol is intended to include all pharmaceutically acceptable salts thereof, including for example, indacaterol maleate and indacaterol hydrochloride. In one embodiment of the present invention, the β₂-agonist is selected from a N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide or a salt thereof,

a N-[2-(Diethylamino)ethyl]-N-(2-{[2-(4-hydroxy-2-oxo-2,3-dihydro-1,3-benzothiazol-7-yl)ethyl]amino}ethyl)-3-[2-(3-chlorophenyl)ethoxy]propanamide or a salt thereof, or a 7-[(1R)-2-({2-[(3-{[2-(2-Chlorophenyl)ethyl]amino}propyl)-thio]ethyl}amino)-1-hydroxyethyl]-4-hydroxy-1,3-benzothiazol-2(3H)-one or a salt thereof.

Thus, one embodiment of the invention relates to a combination of a compound according to formula (I) as defined above (i.e. any one of the compounds falling within the scope of formula (I) as defined above or any one of the specific compounds or salts or polymorphic forms of compounds of formula (I) mentioned above) or a pharmaceutically acceptable salt thereof, and a glucocorticoid receptor agonist. In one embodiment the glucocorticoid receptor agonist is budesonide.

Another embodiment of the invention relates to a combination of a compound according to formula (I) as defined above (i.e. any one of the compounds falling within the scope of formula (I) as defined above or any one of the specific compounds or salts or polymorphic forms of compounds of formula (I) mentioned above) or a pharmaceutically acceptable salt thereof and a β₂-agonist. Yet another embodiment of the invention relates to a combination of a compound according to formula (I) as defined above (i.e. any one of the compounds falling within the scope of formula (I) as defined above or any one of the specific compounds or salts or polymorphic forms of compounds of formula (I) mentioned above) or a pharmaceutically acceptable salt thereof with a glucocorticoid receptor agonist and a β₂-agonist. In another embodiment the glucocorticoid receptor agonist is budesonide. In yet a further embodiment β₂-agonist is selected from any of formoterol, indacaterol or a N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide or a salt thereof, a N-[2-(Diethylamino)ethyl]-N-(2-{[2-(4-hydroxy-2-oxo-2,3-dihydro-1,3-benzothiazol-7-yl)ethyl]amino}ethyl)-3-[2-(3-chlorophenyl)ethoxy]propanamide or a salt thereof, or a 7-[(1R)-2-({2-[(3-{[2-(2-Chlorophenyl)ethyl]amino}propyl)-thio]ethyl}amino)-1-hydroxyethyl]-4-hydroxy-1,3-benzothiazol-2(3H)-one or a salt thereof.

Pharmaceutical Compositions

The active ingredients of the present invention may be administered by oral or parenteral (e.g. intravenous, subcutaneous, intramuscular or intraarticular) administration using conventional systemic dosage forms, such as tablets, capsules, pills, powders, aqueous or oily solutions or suspensions, emulsions and sterile injectable aqueous or oily solutions or suspensions. The active ingredients may also be administered topically (e.g. to the lung and/or airways) in the form of solutions, suspensions, aerosols and dry powder compositions. These dosage forms will usually include one or more pharmaceutically acceptable ingredients which may be selected, for example, from adjuvants, carriers, binders, lubricants, diluents, stabilising agents, buffering agents, emulsifying agents, viscosity-regulating agents, surfactants, preservatives, flavourings and colorants. As will be understood by those skilled in the art, the most appropriate method of administering the active ingredients is dependent on a number of factors.

One embodiment relates to a pharmaceutical composition comprising, in admixture, a first active ingredient, which is a compound of formula (I) as defined above (i.e. any one of the compounds falling within the scope of formula (I) as defined above or any one of the specific compounds or salts or polymorphic forms of compounds of formula (I) mentioned above) or a pharmaceutically acceptable salt thereof and a second active ingredient, which is a glucocorticoid receptor agonist as defined above, and optionally a third active ingredient, which is a β₂ agonist as defined above, with pharmaceutically acceptable adjuvants, diluents and/or carriers.

In another embodiment the glucocorticoid receptor agonist is budesonide.

In yet a further embodiment β₂-agonist is selected from any of formoterol, indacaterol or a N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide or a salt thereof,

a N-[2-(Diethylamino)ethyl]-N-(2-{[2-(4-hydroxy-2-oxo-2,3-dihydro-1,3-benzothiazol-7-yl)ethyl]amino}ethyl)-3-[2-(3-chlorophenyl)ethoxy]propanamide or a salt thereof, or

a 7-[(1R)-2-({2-[(3-{[2-(2-Chlorophenyl)ethyl]amino}propyl)-thio]ethyl}amino)-1-hydroxyethyl]-4-hydroxy-1,3-benzothiazol-2(3H)-one or a salt thereof.

In one embodiment of the present invention the active ingredients are administered via separate pharmaceutical compositions.

One embodiment of the present invention provides a kit comprising a composition of a first active ingredient, which is a compound of formula (I) as defined above (i.e. any one of the compounds falling within the scope of formula (I) as defined above or any one of the specific compounds or salts or polymorphic forms of compounds of formula (I) mentioned above) or a pharmaceutically acceptable salt thereof, and a composition of a second active ingredient, which is a glucocorticoid receptor agonist as defined above, and optionally a composition of a third active ingredient, which is a β₂ agonist as defined above, and optionally instructions for the simultaneous, sequential or separate administration of the compositions to a patient in need thereof.

In another embodiment the glucocorticoid receptor agonist is budesonide.

In yet a further embodiment β₂-agonist is selected from any of formoterol, indacaterol or a N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide or a salt thereof,

a N-[2-(Diethylamino)ethyl]-N-(2-{[2-(4-hydroxy-2-oxo-2,3-dihydro-1,3-benzothiazol-7-yl)ethyl]amino}ethyl)-3-[2-(3-chlorophenyl)ethoxy]propanamide or a salt thereof, or

a 7-[(1R)-2-({2-[(3-{[2-(2-Chlorophenyl)ethyl]amino}propyl)-thio]ethyl}amino)-1-hydroxyethyl]-4-hydroxy-1,3-benzothiazol-2(3H)-one or a salt thereof.

A pharmaceutical composition of the present invention where the active ingredients are in admixture may be prepared by mixing the first active ingredient and the second active ingredient, and optionally a third active ingredient, with a pharmaceutically acceptable adjuvant, diluent or carrier. Therefore, in a further aspect of the present invention there is provided a process for the preparation of a pharmaceutical composition, which comprises mixing a compound of formula (I), as defined above (i.e. any one of the compounds falling within the scope of formula (I) as defined above or any one of the compounds or salts or polymorphs of compounds of formula (I) mentioned above) or a pharmaceutically acceptable salt thereof, with a second active ingredient, which is a glucocorticoid receptor agonist as defined above, and optionally a third active ingredient, which is a β₂ agonist as defined above, and a pharmaceutically acceptable adjuvant, diluent or carrier.

It will be understood that the therapeutic dose of each active ingredient administered in accordance with the present invention will vary depending upon the particular active ingredient employed, the mode by which the active ingredient is to be administered, and the condition or disorder to be treated.

In one embodiment of the present invention, the first and second active ingredients of the present invention are each administered by inhalation. In this embodiment, the active ingredients may be inhaled simultaneously (that is, the active ingredients are in admixture). In another embodiment the active ingredients may be inhaled sequentially. Or in a further embodiment the active ingredients may be inhaled separately.

The active ingredients are conveniently administered via inhalation (e.g. topically to the lung and/or airways) in the form of solutions, suspensions, aerosols or dry powder compositions. Administration may be by inhalation, orally or intranasally. The active ingredients are preferably adapted to be administered, either together or individually, from a dry powder inhaler, pressurised metered dose inhaler, or a nebuliser.

The active ingredients may be used in admixture with one or more pharmaceutically acceptable additives, diluents or carriers. Examples of suitable diluents or carriers include lactose (e.g. the monohydrate), dextran, mannitol or glucose.

Metered dose inhaler devices may be used to administer the active ingredients, dispersed in a suitable propellant and with or without additional excipients such as ethanol, a surfactant, a lubricant, an anti-oxidant or a stabilising agent. Suitable propellants include hydrocarbon, chlorofluorocarbon and hydrofluoroalkane (e.g. heptafluoroalkane) propellants, or mixtures of any such propellants. Preferred propellants are P134a and P227, each of which may be used alone or in combination with other propellants and/or surfactant and/or other excipients. Nebulised aqueous suspensions, solutions may also be employed, with or without a suitable pH and/or tonicity adjustment, either as a unit-dose or multi-dose compositions.

Dry powder inhalers may be used to administer the active ingredients, alone or in combination with a pharmaceutically acceptable carrier, in the later case either as a finely divided powder or as an ordered mixture. The dry powder inhaler may be single dose or multi-dose and may utilise a dry powder or a powder-containing capsule.

When the active ingredients are adapted to be administered, either together or individually, via a nebuliser they may be in the form of a nebulised aqueous suspension or solution, with or without a suitable pH or tonicity adjustment, either as a single dose or multidose device.

Metered dose inhaler, nebuliser and dry powder inhaler devices are well known and a variety of such devices are available.

In one embodiment, the present invention provides a pharmaceutical product comprising, in combination, a first active ingredient which is a compound of formula (I), as defined above (i.e. any one of the compounds falling within the scope of formula (I) as defined above or any one of the specific compounds or salts or polymorphic forms of compounds of formula (I) mentioned above) or a pharmaceutically acceptable salt thereof, and a second active ingredient, which is a glucocorticoid receptor agonist, as defined above, wherein each active ingredient is formulated for inhaled administration.

In another embodiment of the present invention, the first active ingredient, which is a compound of formula (I), as defined above (i.e. any one of the compounds falling within the scope of formula (I) as defined above or any one of the specific compounds or salts or polymorphic forms of compounds of formula (I) mentioned above) or a pharmaceutically acceptable salt thereof, may be formulated for oral administration and the second active ingredient(s), which is a glucocorticoid receptor agonist, as defined above, may be formulated for inhaled administration.

In yet another embodiment of the present invention, the first active ingredient, which is a compound of formula (I), as defined above (i.e. any one of the compounds falling within the scope of formula (I) as defined above or any one of the specific compounds or salts or polymorphic forms of compounds of formula (I) mentioned above) or a pharmaceutically acceptable salt thereof may be formulated for inhalated administration and the second active ingredient(s), which is a glucocorticoid receptor agonist, as defined above, may be formulated for oral administration.

In yet a further embodiment of the present invention, the first active ingredient, which is a compound of formula (I), as defined above (i.e. any one of the compounds falling within the scope of formula (I) as defined above or any one of the specific compounds or salts or polymorphic forms of compounds of formula (I) mentioned above) or a pharmaceutically acceptable salt thereof, and the second active ingredient(s), which is a glucocorticoid receptor agonist, as defined above, wherein each active ingredient is formulated for oral administration.

Medical Use

The use of a compound of formula (I) is contemplated to demonstrate particular effects when used in combination with a glucocorticoid receptor agonist, and in particular in combination with budesonide. For example, in vivo animal experiments indicate that a combination of a glucocorticoid receptor agonist and a compound of formula (I), at dose levels where neither component alone significantly affects lung inflammation, in combination give significant reduction of inflammatory cell influx. The reduction in cell influx for the combination is contemplated to be greater than that expected from the additive effect of the two ingredients. This synergistic effect ould be used, for example, to lower the therapeutic dose of glucocorticoid receptor agonist, or at the same dose, achieve enhanced efficacy on inflammation in comparison to the use of the glucocorticoid receptor agonist alone. The synergistic effect can be particularly advantageous where lower doses of the glucocorticoid receptor agonist are desirable, for example in individuals that have acquired resistance to such a glucocorticoid receptor agonist.

Examples of conditions and diseases, which may be treated using the combination of the invention are, but not limited to, airways diseases/respiratory including chronic obstructive pulmonary disease (COPD) such as irreversible COPD; asthma, such as bronchial, allergic, intrinsic, extrinsic and dust asthma, particularly chronic or inveterate asthma (e.g. late asthma and airways hyper-responsiveness); bronchitis; acute, allergic, atrophic rhinitis and chronic rhinitis including rhinitis caseosa, hypertrophic rhinitis, rhinitis purulenta, rhinitis sicca and rhinitis medicamentosa; membranous rhinitis including croupous, fibrinous and pseudomembranous rhinitis and scrofoulous rhinitis; seasonal rhinitis including rhinitis nervosa (hay fever) and vasomotor rhinitis; sarcoidosis, farmer's lung and related diseases, fibroid lung and idiopathic interstitial pneumonia.

The compound of formula (I), as defined above (i.e. any one of the compounds falling within the scope of formula (I) as defined above or any one of the specific compounds or salts or polymorphic forms of compounds of formula (I) mentioned above) or a pharmaceutically acceptable salt thereof (first active ingredient) and the glucocorticoid receptor agonist as defined above or a pharmaceutically acceptable salt thereof, (second active ingredient of the present invention) may be administered simultaneously, sequentially or separately to treat airway diseases. By sequential it is meant that the active ingredients are administered, in any order, one immediately after the other. They still have the desired effect if they are administered less than 4 hours apart, more conveniently less than two hours apart, more conveniently less than 30 minutes apart and most conveniently less than 10 minutes apart.

Throughout the specification, the amount of the active ingredients used relate to unit doses unless explicitly defined differently.

When administered via inhalation the dose of the first active ingredient (compound of formula (I) or a pharmaceutically acceptable salt thereof), will generally be in the range of from 0.1 μg to 10000 μg, 0.1 to 5000 μg, 0.1 to 1000 μg, 0.1 to 500 μg, 0.1 μg to 200 μg, 0.1 to 200 μg, 0.1 to 100 μg, 0.1 to 50 μg, 5 μg to 5000 μg, 5 to 1000 82 g, 5 to 500 μg, 5 μg to 200 μg, 5 to 100 μg, 5 to 50 μg, 10 to 5000 μg, 10 to 1000 μg, 10 to 500 μg, 10 to 200 μg, 10 to 100 μg, 10 to 50 μg, 20 to 5000 μg, 20 to 1000 μg, 20 to 500 μg, 20 to 200 μg, 20 to 100 μg, 20 to 50 μg, 50 to 5000 μg, 50 to 1000 μg, 50 to 500 μg, 50 to 200 μg, 50 to 100 μg, 100 to 5000 μg, 100 to 1000 μg or 100 to 500 μg.

In one embodiment, the amount of the first active ingredient used is in the range 1 μg to 200 μg, and that of the second ctive ingredient in the range 1 μg to 200 μg.

When administered via inhalation the dose of the second active ingredient (glucocorticoid receptor agonist), will generally be in the range of from 0.1 microgram (m) to 1000 μg, 0.1 to 500 μg, 0.1 to 200 μg, 0.1 to 100 μg, 0.1 to 50 μg, 0.1 to 5 μg, 5 to 1000 μg, 5 to 500 μg, 5 to 200 μg, 5 to 50 μg, 5 to 10 μg, 10 to 1000 μg, 10 to 500 μg, 10 to 200 μg, 10 to 100 μg, 10 to 50 μg, 20 to 1000 μg, 20 to 500 μg, 20 to 200 μg, 20 to 100 μg, 20 to 50 μg, 50 to 1000 μg, 50 to 500 μg, 50 to 200 μg, 50 to 100 μg, 100 to 1000 to 500 μg.

When administered via inhalation the dose of the third active ingredient (β₂ agonist), will generally be in the range of from 0.1 microgram (m) to 1000 μg, 0.1 to 500 μg, 0.1 to 200 μg, 0.1 to 100 μg, 0.1 to 50 μg, 0.1 to 5 μg, 5 to 1000 μg, 5 to 500 μg, 5 to 200 μg, 5 to 50 μg, 5 to 10 μg, 10 to 1000 μg, 10 to 500 μg, 10 to 200 μg, 10 to 100 μg, 10 to 50 μg, 20 to 1000 μg, 20 to 500 μg, 20 to 200 μg, 20 to 100 μg, 20 to 50 μg, 50 to 1000 μg, 50 to 500 μg, 50 to 200 μg, 50 to 100 μg, 100 to 1000 μg, or 100 to 500 μg.

The molar ratio of the second active ingredient to the first active ingredient in a dose may typically be in the range of 300:1 to 1:300. In one embodiment the ratio is in the range of from 100:1 to 1:100. In another embodiment the ratio is in the range of from 50:1 to 1:50. In a further embodiment the ratio is in the range of from 10:1 to 1:10. In yet another embodiment the ratio is in the range of from 5:1 to 1:5.

In one embodiment the ratio is in the range of 1:10 to 1:60. In a further embodiment the ratio is in the range of 1:40 to 1:60. In another embodiment the ratio is in the range of 1:15 to 1:20.

When the third active ingredient is added the molar ratio of the third active ingredient to the second active ingredient to the first active ingredient in a dose may typically be in the range of 100:300:1 to 10:300:1 or 100:1:300 or 10:1:300.

The doses of the first, second and optionally third active ingredients will generally be administered from 1 to 4 times a day, conveniently once or twice a day, and most conveniently once a day.

The present invention further provides a pharmaceutical product, kit or pharmaceutical composition comprising the combination according to the present invention for simultaneous, sequential or separate use in therapy.

The present invention further provides the use of a pharmaceutical product, kit or pharmaceutical composition, which comprises:

(a) a (therapeutically effective) dose of a first active ingredient, which is a compound of formula (I), as defined above (i.e. any one of the compounds falling within the scope of formula (I) as defined above or any one of the specific compounds or salts or polymorphic forms of compounds of formula (I) mentioned above) or a pharmaceutically acceptable salt thereof and

(b) a (therapeutically effective) dose of a second active ingredient, which is a glucocorticoid receptor agonist as defined above or a pharmaceutically acceptable salt thereof and optionally,

(c) a (therapeutically effective) dose of a third active ingredient, which is a β₂ agonist as defined above, in the manufacture of a medicament for the treatment of airway diseases.

In another embodiment the glucocorticoid receptor agonist is budesonide.

In yet a further embodiment β₂-agonist is selected from any of formoterol, indacaterol or a N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide or a salt thereof, a N-[2-(Diethylamino)ethyl]-N-(2-{[2-(4-hydroxy-2-oxo-2,3-dihydro-1,3-benzothiazol-7-yl)ethyl]amino}ethyl)-3-[2-(3-chlorophenyl)ethoxy]propanamide or a salt thereof, or a 7-[(1R)-2-({2-[(3-{[2-(2-Chlorophenyl)ethyl]amino}propyl)-thio]ethyl}amino)-1-hydroxyethyl]-4-hydroxy-1,3-benzothiazol-2(3H)-one or a salt thereof

The present invention further provides the use of a pharmaceutical product, kit or pharmaceutical composition, which comprises:

(a) a (therapeutically effective) dose of a first active ingredient, which is a compound of formula (I), as defined above (i.e. any one of the compounds falling within the scope of formula (I) as defined above or any one of the specific compounds or salts or polymorphic forms of compounds of formula (I) mentioned above) or a pharmaceutically acceptable salt thereof and

(b) a (therapeutically effective) dose of a second active ingredient, which is a glucocorticoid receptor agonist as defined above or a pharmaceutically acceptable salt thereof and optionally,

(c) a (therapeutically effective) dose of a third active ingredient, which is a β₂ agonist as defined above, provided the agonist is not selected from

a N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide or a salt thereof,

a N-[2-(Diethylamino)ethyl]-N-(2-{[2-(4-hydroxy-2-oxo-2,3-dihydro-1,3-benzothiazol-7-yl)ethyl]amino}ethyl)-3-[2-(3-chlorophenyl)ethoxy]propanamide or a salt thereof, or

a 7-[(1R)-2-({2-[(3-{[2-(2-Chlorophenyl)ethyl]amino}propyl)-thio]ethyl}amino)-1-hydroxyethyl]-4-hydroxy-1,3-benzothiazol-2(3H)-one or a salt thereof, in the manufacture of a medicament for the treatment of chronic obstructive pulmonary disease or asthma, or any other disorder mentioned above.

The present invention still further provides a method of treating airway diseases, or chronic obstructive pulmonary disease or asthma, or any other disorder mentioned above which comprises simultaneously, sequentially or separately administering:

(a) a (therapeutically effective) dose of a first active ingredient, which is a compound of formula (I), as defined above (i.e. any one of the compounds falling within the scope of formula (I) as defined above or any one of the specific compounds or salts or polymorphic forms of compounds of formula (I) mentioned above) or a pharmaceutically acceptable salt thereof and

(b) a (therapeutically effective) dose of a second active ingredient, which is a glucocorticoid receptor agonist or a pharmaceutically acceptable salt thereof and optionally,

(c) a (therapeutically effective) dose of a third active ingredient, which is a β₂ agonist as defined above, provided the agonist is not selected from

a N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide or a salt thereof,

a N-[2-(Diethylamino)ethyl]-N-(2-{[2-(4-hydroxy-2-oxo-2,3-dihydro-1,3-benzothiazol-7-yl)ethyl]amino}ethyl)-3-[2-(3-chlorophenyl)ethoxy]propanamide or a salt thereof, or

a 7-[(1R)-2-({2-[(3-{[2-(2-Chlorophenyl)ethyl]amino}propyl)-thio]ethyl}amino)-1-hydroxyethyl]-4-hydroxy-1,3-benzothiazol-2(3H)-one or a salt thereof to a patient in need thereof

In another embodiment the glucocorticoid receptor agonist is budesonide.

In yet a further embodiment β₂-agonist is selected from any of formoterol, indacaterol or a N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide or a salt thereof,

a N-[2-(Diethylamino)ethyl]-N-(2-{[2-(4-hydroxy-2-oxo-2,3-dihydro-1,3-benzothiazol-7-yl)ethyl]amino}ethyl)-3-[2-(3-chlorophenyl)ethoxy]propanamide or a salt thereof, or

a 7-[(1R)-2-({2-[(3-{[2-(2-Chlorophenyl)ethyl]amino}propyl)-thio]ethyl}amino)-1-hydroxyethyl]-4-hydroxy-1,3-benzothiazol-2(3H)-one or a salt thereof.

One embodiment relates to a pharmaceutical product, use or method described above wherein (a) a first active ingredient is 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid or a pharmaceutically acceptable salt thereof, and

(b) a second active ingredient is budesonide.

One embodiment relates to a pharmaceutical product, use or method described above wherein (a) a first active ingredient is 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid or a pharmaceutically acceptable salt thereof,

(b) a second active ingredient is budesonide, and

(c) a third active ingredient is selected from any of formoterol, indacaterol or a N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide or a salt thereof,

a N-[2-(Diethylamino)ethyl]-N-(2-{[2-(4-hydroxy-2-oxo-2,3-dihydro-1,3-benzothiazol-7-yl)ethyl]amino}ethyl)-3-[2-(3-chlorophenyl)ethoxy]propanamide or a salt thereof, or

a 7-[(1R)-2-({2-[(3-{[2-(2-Chlorophenyl)ethyl]amino}propyl)-thio]ethyl}amino)-1-hydroxyethyl]-4-hydroxy-1,3-benzothiazol-2(3H)-one or a salt thereof.

One embodiment of the invention relates to the combination as described above wherein a phospodiesterase (PDE) inhibitor or a muscarinic antagonist is excluded from the combination of the invention.

In the context of the present specification, the term “therapy” also includes “prophylaxis” unless there are specific indications to the contrary. The terms “therapeutic” and “therapeutically” should be construed accordingly. Prophylaxis is expected to be particularly relevant to the treatment of persons who have suffered a previous episode of, or are otherwise considered to be at increased risk of, the condition or disorder in question. Persons at risk of developing a particular condition or disorder generally include those having a family history of the condition or disorder, or those who have been identified by genetic testing or screening to be particularly susceptible to developing the condition or disorder.

The term “disease”, unless stated otherwise, has the same meaning as the terms “condition” and “disorder” and are used interchangeably throughout the description and claims.

The term “agent” and “ingredient” means the compounds comprised in the combination of the present invention, i.e. an CCR1 antagonist or a glucocorticoid receptor agonist .

### DESCRIPTION OF THE FIGURES

FIG. 1 shows the results of a cell influx experiment in LPS-challenged rats using a combination of the present invention.

### EXAMPLES

The present invention will now be further understood by reference to the following illustrative examples.

The following abbreviations are used:

APCI-MS Atmospheric Pressure Chemical Ionisation Mass Spectroscopy;

DCM Dichloromethane

DIEA N, N-Diisopropylethylamine;

DMF N, N-Dimethylformamide

DMSO Dimethylsulfoxide;

HPLC High Performance Liquid Chromatography;

LC/MS Liquid Column Chromatography/Mass Spectroscopy;

TFA Trifluoroacetic acid;

THF Tetrahydrofuran

EtOAc Ethylacetate

Eq equivalent

TMS Tetramethyl silane

General Methods

¹H NMR and ¹³C NMR spectra were recorded on a Varian Inova 400 MHz or a Varian Mercury-VX 300 MHz instrument. The central peaks of chloroform-d₃ (δ_{H} 7.27 ppm), dimethylsulfoxide-d₆ (δ_{H} 2.50 ppm), acetonitrile-d₃ (δ_{H} 1.95 ppm) or methanol-d₄ (δ_{H} 3.31 ppm) were used as internal references. Flash chromatography was carried out using silica gel (0.040-0.063 mm, Merck). Unless stated otherwise, starting materials were commercially available. All solvents and commercial reagents were of laboratory grade and were used as received.

The following method was used for LC/MS analysis:

Instrument Agilent 1100; Column Waters Symmetry 2.1×30 mm; Mass APCI; Flow rate 0.7 ml/min; Wavelength 254 nm; Solvent A: water+0.1% TFA; Solvent B: acetonitrile+0.1% TFA; Gradient 15-95%/B 2.7 min, 95% B 0.3 min.

The following method was used for LC analysis:

Method A. Instrument Agilent 1100; Column: Kromasil C18 100×3 mm, 5μ particle size,

Solvent A: 0.1% TFA/water, Solvent B: 0.08% TFA/acetonitrile Flow: 1 ml/min,

Gradient 10-100% B 20 min, 100% B 1 min. Absorption was measured at 220, 254 and 280 nm.

Method B. Instrument Agilent 1100; Column: XTerra C8, 100×3 mm, 5μ particle size,

Solvent A: 15 mM NH₃/water, Solvent B: acetonitrile Flow: 1 ml/min, Gradient 10-100% B 20 min, 100% B 1 min. Absorption was measured at 220, 254 and 280 nm.

### Example 1

### 5-Chloro-2-{[(2S)-3-(5-chloro-1′H,3H-spiro[1-benzofuran-2,4′-piperidin]-1′-yl)-2-hydroxypropyl]oxy}-4-(cyanomethoxy)benzoic acid trifluoroacetate (salt)

A mixture of 5-chloro-2-{[(2S)-3-(5-chloro-l′H,3H-spiro[1-benzofuran-2,4′-piperidin]-1′-yl)-2-hydroxypropyl]oxy}-4-hydroxybenzoic acid (29 mg), bromoacetonitrile (6 mg), and cesium carbonate (65 mg) in DMF (1 ml) was stirred at room temperature for 1 h. Then the inorganic precipitate was removed, and the product purified by HPLC yielding 25 mg (TFA salt) of the subtitled compound as white solid.

¹H-NMR (d₆-acetone, 400 MHz): δ 7.87 (s, 1H), 7.23 (s, 1H), 7.14 (dd, J=8.5, 2.2 Hz, 1H), 7.07 (s, 1H), 6.77 (d, J=8.6 Hz, 1H), 5.15 (s, 2H), 4.63 (m, 1H), 4.26 (dd, j=9.8, 4.3 Hz, 1H), 4.14 (dd, J=9.9, 5.9 Hz, 1H), 3.87 (br.s, 2H), 3.71 (br.d, J=11.6 Hz, 11.6 Hz, 1H), 3.58 (dd, J=13.4, 8.9 Hz, 2H), 3.55 (br.s, 1H), 3.20 (s, 2H), 2.49-2.18 (m, 4H); APCI-MS: m/z 507(M⁺).

### Example 2

### 2-{[(2S)-3-(5-chloro-1′H,3H-spiro[1-benzofuran-2,4′-piperidin]-1′-yl)-2-hydroxypropyl]oxy}-5-chloro-4-(2,2-difluoroethoxy)benzoic acid trifluoroacetate (salt)

### Step 1 Benzoic acid, 2-{[(2S)-3-(5-chloro-1′H,3H-spiro[1-benzofuran-2,4′-piperidin]-1′-yl)-2-hydroxypropyl]oxy}-5-chloro-4-(2,2-difluoroethoxy)-, ethyl ester

A solution of benzoic acid, 5-chloro-2-[(2S)-3-(5-chlorospiro[benzofuran-2(3H),4′-piperidin]-1′-yl)-2-hydroxypropoxy]-4-[(4-methoxyphenyl)methoxy]-, ethyl ester (190 mg) and ceric ammonium nitrate (844 mg) in a mixture of acetonitrile (3.5 ml) and water (1.5 ml) was stirred at room temperature for 45 min. The mixture was quenched with a saturated solution of NaHCO₃ (aq) and extracted with DCM. The combined organic layers are dried over anhydrous sodium sulphate, filtrated and the solvents are removed in vacuo. Part of the residue (50 mg) was redissolved in DMF. To the solution are added 2-bromo-1,1-difluoroethane (4 eq) and cesium carbonate (2 eq). The reaction was stirred at 70° C. for 18 h, partitioned between EtOAc and water and the aquaous layer was extracted with EtOAc. The combined organic layers are dried over anhydrous sodium sulphate, filtrated and removed in vacuo. The residue was purified by HPLC (X-Terra) to give the subtitled compound.

### Step 2 Benzoic acid, 2-{[(2S)-3-(5-chloro-1′H,3H-spiro[1-benzofuran-2,4′-piperidin]-1′-yl)-2-hydroxypropyl]oxy}-5-chloro-4-(2,2-difluoroethoxy)benzoic acid trifluoroacetate (salt)

Benzoic acid, 2-{[(2S)-3-(5-chloro-1′H,3H-spiro[1-benzofuran-2,4′-piperidin]-1′-yl)-2-hydroxypropyl]oxy}-5-chloro-4-(2,2-difluoroethoxy)-, ethyl ester (20 mg) was dissolved in ethanol (2 ml). An aqueous solution of NaOH (2 M, 2 ml) was added, and the mixture was heated at 80° C. for 30 min. The solution was concentrated in vacuo, acidified with TFA, and purified by HPLC yielding 10 mg of the titled compound.

¹H-NMR (CD₃OD, 500 MHz): δ 7.57 (s, 1H), 7.14 (s, 1H), 7.42 (m, 1H), 7.78 (s, 1H), 6.66 (d, J=8.6 Hz, 1H), 6.33-9.10 (m, 1H), 4.37-4.31 (m, 2H), 4.20-4.15 (m, 2H), 4.03-4.99 (m, 1H), 3.02 (s 2H), 2.89-2.68 (m, 6H), 1.96-1.89 (m, 4H).

### Example 3

### 5-Chloro-2-{[(2S)-3-(5-chloro-1′H,3H-spiro[1-benzofuran-2,4′-piperidin]-1′-yl)-2-hydroxypropyl]oxy}-4-(3,3,3-trifluoropropoxy)benzoic acid trifluoroacetate (salt)

### Step 1: Methyl 5-chloro-4-[(4-methoxybenzyl)oxy]-2-[(2S)-oxiran-2-ylmethoxy]benzoate

A mixture of methyl 5-chloro-2-hydroxy-4-[(4-methoxybenzyl)oxy]benzoate (654 mg, (2S)-oxiran-2-ylmethyl-3-nitrobenzenesulfonate (518 mg), and Cs₂CO₃ (986 mg) in DMF (10 ml) was stirred at room temperature for 18 h. EtOAc (100 ml) was added, the organic layer was washed with water (2×50 ml). The organic layer was dried with sodium sulphate, filtrated and the solvent removed in vacuo yielding 738 mg of the subtitled compound. ¹H-NMR (CDCl₃, 400 MHz): δ 7.86 (d, J=8.6 Hz, 1H), 7.35 (m, 2H), 6.93 (m, 2H), 6.59 (m, 2H), 5.02 (s, 2H), 4.30 (dd, J=11.2, 2.9 Hz, 1H), 4.06 (dd, J=11.2, 4.8 Hz, 1H), 3.86 (s, 3H), 3.83 (d, J=1.8 Hz, 3H), 3.39 (m, 1H), 2.97-2.88 (m, 2H). APCI-MS: m/z 345 (MH⁻).

### Step 2: Methyl 5-chloro-2-{[(2S)-3-(5-chloro-1′H,3H-spiro[1-benzofuran-2,4′-piperidin]-1′-yl)-2-hydroxypropyl]oxy}-4-hydroxybenzoate

A mixture of 5-chloro-3H-spiro[1-benzofuran-2,4′-piperidine] (436 mg), methyl 5-chloro-4-[(4-methoxybenzyl)oxy]-2-[(2S)-oxiran-2-ylmethoxy]benzoate (738 g) in dry ethanol (10 ml) was stirred at 80° C. for 18 h. Then the solvent was removed in vacuo and the residue dissolved in dichloromethane (20 ml). TFA (3 ml) was addded, and the reaction was stirredat room temperature for 2.5 h. The solvent was removed in vacuo, and the residue purified by HPLC to afford the subtitle compound as colourless solid, 256 mg (TFA salt). APCI-MS: m/z 482 (MH⁻).

### Step 3: Methyl 5-chloro-2-{[(2S)-3-(5-chloro-1′H,3H-spiro[1-benzofuran-2,4′-piperidin]-1′-yl)-2-hydroxypropyl]oxy}-4-(3,3,3-trifluoropropoxy)benzoate

A mixture of methyl 5-chloro-2-{[(2S)-3-(5-chloro-1′H,3H-spiro[1-benzofuran-2,4′-piperidin]-1′-yl)-2-hydroxypropyl]oxy}-4-hydroxybenzoate (119 mg), 3-bromo-1,1,1-trifluoropropane (70 mg), and Cs₂CO₃ (263 mg) was stirred at room temperature for 18 h . A second portion of 3-bromo-1,1,1-trifluoropropane (0.5 ml) and Cs₂CO₃ (760 mg) were added, and stirring was continued for 168 h. The inorganic precipitate was removed by filtration, and the product purified by HPLC yielding 36 mg of the subtitled compound as the TFA salt.

¹H-NMR (d₆-acetone, 400 MHz): δ 7.85 (s, 1H), 7.24 (s, 1H), 7.14 (dd, J=8.5, 2.2 Hz, 1H), 7.05 (s, 1H), 6.78 (d, J=8.5 Hz, 1H), 4.60 (m, 1H), 4.53 (t, J=6.1 Hz, 2H), 4.34 (dd, J=9.5, 4.6 Hz, 1H), 4.18 (dd, J=9.4, 6.4 Hz, 1H), 3.86 (s, 3H), 3.84 (br.s, 1H), 3.67 (m, 1H), 3.49 (dd, J=13.4, 9.3Hz, 4H), 3.19 (s, 2H), 2.88 (m, 2H), 2.47-2.18 (m, 4H) APCI-MS: m/z 578 (WO.

### Step 4

Methyl 5-chloro-2-{[(2S)-3-(5-chloro-1′H,3H-spiro[1-benzofuran-2,4′-piperidin]-1′-yl)- 2-hydroxypropyl]oxy}-4-(3,3,3-trifluoropropoxy)benzoate (33 mg) was dissolved in ethanol (2 ml). An aquaous solution of NaOH (2 M, 2 ml) was added, and the mixture was heated at 80° C. for 30 min. The solution was concentrated in vacuo, acidified with TFA, and the product purified by HPLC yielding 27 mg of the title compound as the TFA salt).

¹H-NMR (d₆-acetone, 400 MHz): δ 7.92 (s, 1H), 7.23 (s, 1H), 7.13 (dd, J=8.5, 2.2 Hz, 1H), 7.06 (s, 1H), 6.77 (d, J=8.6 Hz, 1H), 4.64 (dd, J=13.7, 8.5 Hz, 1H), 4.53 (t, J=6.1 Hz, 2H), 4.40 (dd, J=9.6, 4.8 Hz, 1H), 4.26 (dd, J=9.6, 6.1 Hz, 1H), 3.83-3.30 (m, 6H), 3.20 (s, 2H), 2.87 (m, 2H), 2.45-2.16 (m, 4H).

### Example 4

### N-(2-{3-[5-chloro-1′H,3H-spiro[-benzofuran-2,4′-piperidin]-1′-yl]propoxy}phenyl)acetamide trifluoroacetate (salt)

A mixture of N-(2-hydroxyphenyl)acetamide (1 mmol), 1-bromo-3-chloropropane (1 mmol) and cesium carbonate (1.5 eq) in DMF (4 ml) was stirred at room temperature for 6 h. Subsequently 5-chloro-3H-spiro[1-benzofuran-2,4′-piperidine] (1 eq) and a second portion of cesium carbonate (1.5 eq) are added and the reaction was stirred at 70° C. for 18 h. The reaction mixture was extracted between EtOAc and water, dried over anhydrous sodium sulfate, filtrated and the solvent removed in vacuo. The residue was subsequently purified over silica (flash) and HPLC (water/acetonitrile with 0.1% TFA), providing 30 mg of the titled compound as a white solid.

1H NMR (CD₃OD) δ 7.65 (m, 1H), 7.20-7.05 (m, 4H), 6.97 (m, 1H), 6.75 (m, 1H), 4.22-4.19 (m, 2H), 3.72-3.21 (m, 6H), 3.12 (m, 2H), 2.34-2.05 (m, 6H), 2.18 (s, 3H); APCI-MS m/z 415(M⁺).

### Example 5

### Methyl3-(2-{[(2S)-3-(5-chloro-1′H3H-spiro[1′-benzofuran-2,4′-piperidin]-1′-yl)-2-hydroxypropyl]oxy}-4-fluorophenyl)propanoic acid trifluo acetic acid salt

### Step 1. Methyl (2E)-3-(4-fluoro-2-hydroxyphenyl)acrylate

To an ice-cooled solution of 4-fluoro-2-hydroxybenzaldehyde (420 mg) in THF (9 ml) a solution of methyl(triphenylphosphoranylidene)acetate (1 g) in dichloromethane (6 ml) was added dropwise, after which the reaction mixture was stirred at room temperature for 24 h. The solvents were removed in vacuo and the residue was purified by silica gel flash chromatography to give the subtitled compound (545 mg, 93%).

¹H-NMR (DMSO-d₆, 400 MHz): δ 11.00 (br.s, 1H); 7.81 (d, J=16.1 Hz, 1H); 7.67 (t, J=7.9 Hz, 1H); 6.71-6.54 (m, 2H); 6.59 (d, J=16.1 Hz, 1H); 3.68 (s, 3H).

### Step 2. Methyl 3-(4-fluoro-2-hydroxyphenyl)propanoate

To a solution of methyl (2E)-3-(4-fluoro-2-hydroxyphenyl)acrylate (350 mg) in EtOAc (12 ml), Pt/C (5%, 70 mg) was added and the mixture was hydrogeneted at atmospheric pressure and at room temperature for 8 h. The catalyst was filtered off and the filtrate was concentrated in vacuo to give the subtitled compoud (327 mg, 93%).

¹H-NMR (DMSO-d₆, 400 MHz): δ 9.99 (br.s, 1H); 7.02 (t, J=7.7 Hz, 1H); 6.54 (dd, J=2.6, 10.8 Hz, 1H); 6.51-6.45 (m, 1H); 3.59 (s, 3H); 2.70 (t, J=7.6 Hz, 2H); 2.50 (m, 2H).

### Step 3. Methyl3-(2-{[(2S)-3-(5-chloro-1′H3H-spiro[1-benzofuran-2,4′-peperidinal]-1′-yl)-2-hydroxypropyl]oxy}-4-fluorophenyl)propanoic acid Trifluoroacetic acid salt

A mixture of methyl 3-(4-fluoro-2-hydroxyphenyl)propanoate (56 mg), (2S)-oxiran-2-ylmethyl 3-nitrobenzenesulfonate (73 mg) and Cs₂CO₃ (110 mg) in DMF (2 ml) was stirred at room temperature for 18 h, after which it was partitioned between EtOAc and water. The orgaic layer was dried over Na₂SO₄, filtered and the filtrate was concentrated in vacuo to give crude methyl3-{4-fluoro-2-[(2S)-oxiran-2-ylmethoxy]phenyl}propanoate which was redissolved in methanol (1.5 ml). 5-Chloro-3H-spiro[1-benzofuran-2,4′-piperidine] (64 mg) was added to this solution and the mixture was stirred at 80° C. for 8 h. The volatiles were removed in vacuo and the residue was redissolved in THF (3 ml), aqueous NaOH (165 mg NaOH in 1.5 ml water) was added and the reaction mixture was stirred at 80° C. for 2 h, cooled to 0° C. and pH was adjusted to 2 by addition of aq. TFA. The volatiles were removed in vacuo and the residue was purified by HPLC (10-90% CH₃CN in H₂O containing 0.1% TFA) to give the title compound (100 mg).

¹H-NMR (CD₃OD, 400 MHz): δ 7.22-7.10 (m, 3H); 6.76 (m, 2H); 6.64 (m, 1H); 4.50 (m, 1H); 4.08 (m, 2H); 3.8-3.4 (m, 6H); 3.18 (s, 2H); 2.91 (t, J=7.8 Hz, 2H); 2.58 (m, 2H); 2.30-2.08 (m, 4H). APCI-MS: m/z 464 (MH⁺)

### Example 6

### N-(2-{[(2S)-3-({spiro[indole-2-4′-piperidin]-3(1H)-one}-1′-yl)-2-hydroxypropyl]oxy}-4-hydroxyphenyl)acetamide

### Step 1 Ethyl 4-[(2-bromophenyl)amino]-4-cyanopiperidine-1-carboxylate

Reaction kept under nitrogen. To a solution of 2-bromoaniline (142 mg) and N-carbethoxy-4-piperidone (1 eq) in acetic acid (1 ml) TMS-cyanide (1.05 eq) was added. After 40 h the reaction was quenched with NH₄OH (3 ml) under vigorous stirring. The suspension was filtered through extrelut and the solvent removed in vacuo. The residue was purified over silica, yielding 295 mg (100%) of the subtitled compound.

1H NMR (CD₃OD) δ 7.51 (m, 1H), 7.27 (m, 1H), 7.19 (m, 1H), 6.78 (m, 1H), 4,17 (m, 2H), 3.98 (m, 2H), 3.43 (m, 2H), 2.38 (m, 2H), 1.91 (m, 3H); APCI-MS: m/z 352 (M).

### Step 2 Spiro[indole-2,4′-piperidin]-3(1H)-one

To a solution of ethyl 4-[(2-bromophenyl)amino]-4-cyanopiperidine-1-carboxylate (130 mg) in toluene (3 ml) are added first triethylborane (2.5 eq) and then tri-n-butyltin hydride (4 eq). Air was bubbled through the reaction (0.5 ml). The reaction was stirred at room temperature for 48 h. The mixture was quenched with NHCO₃ (aq) and stirred vigorously before filtration through extrelut. The solvent was removed in vacuo and the yellow oil purified over silica yielding 39 mg (39%) of ethyl 3-imino-1,3-dihydro-1′H-spiro[indole-2,4′-piperidine]-1′-carboxylate (APCI-MS: m/z 274(M⁺)), which was redissolved in 6M HCl (aq) (1.5 ml) and refluxed for 18 h. The reaction was quenched with ice, basified with NH₄OH (aq) and diluted with EtOAc. The aquaous phase was extracted three times with EtOAc. The combined organic layers are dried over sodium sulfate, filtrated and removed in vacuo. The residue was purified over silica, yielding 22 mg of the subtitled compound

1H NMR (CD₃OD) δ 7.50-7.44 (m, 2H), 6.92 (m, 1H), 6.74 (m, 1H); 3.16 (m, 2H), 2.91 (m, 2H), 1.85 (m, 2H), 1.33 (m, 2H); APCI-MS: m/z 203(M⁺).

### Step 3 N-(2-{[(2S)-3-({spiro[indole-2-4′-piperidin]-3(1H)-one}-1′-yl)-2-hydroxypropylloxy}-4-hydroxyphenyl)acetamide

A mixture of spiro[indole-2,4′-piperidin]-3(1H)-one (22 mg) and N-[4-(acetyloxy)-2-[(2S)-oxiranylmethoxy]phenyl)-acetamide (29 mg) in ethanol (1 ml) was heated at 90° C. for 18 h. After removal of the solvent in vacuo, the residue was purified over silica, providing the titled compound as a white solid (27 mg).

1H NMR (CD₃OD) δ 7.50 (m, 1H), 7.42-7.36 (m, 2H), 6.84 (m, 1H), 6.66 (m, 1H), 6.42 (m, 1H), 6.29 (m, 1H), 4.14-3.86 (m, 3H), 2.99 (m, 2H), 2.61-2.36 (m, 4H), 2.06 (s, 3H), 1.93 (m, 2H), 1.30 (m, 2H); APCI-MS: m/z 425(M⁺).

### Example 7

### (2-{[(2S )-3-(5-Chloro-1′H,3H-spiro[1-benzofuran-2,4′-piperidin]-1′-yl)-2-hydroxypropyl]oxy}-4-fluorophenyl)methanesulfonic acid

### Step 1: {4-Fluoro-2-[(2S)-oxiran-2-ylmethoxy]phenyl}methanol

A mixture of 5-fluoro-2-(hydroxymethyl)phenol (71 mg), [(25)-2-methyloxiran-2-yl]methyl 3-nitrobenzenesulfonate (130 mg), and Cs₂CO₃ (1.5 eq) in DMF (3 ml) was stirred at room for 18 h. EtOAc (100 ml)was added. The organic phase was washed with water (2×50 ml), dried with sodium sulphate, filtrated and removed in vacuo, yielding 87 mg of the subtitled compound, which was used without further purification.

¹H-NMR (CDCl₃, 400 MHz): δ 7.26 (m, 1H), 6.69 (dd, J=8.2, 2.4 Hz, 1H), 6.63 (m, §H), 4.67 (dd, J=19.1, 12.7 Hz, 2H), 4.33 (dd, J=11.1, 2.7 Hz, 1H), 3.99 (dd, J=11.1, 5.5 Hz, 1H), 3.38 (m, 1H), 2.94 (t, J =4.5 Hz, 1H), 2.82 (dd, J =4.8, 2.7 Hz, 1H).

### Step 2: (2S)-1-(5-Chloro-1′H,3H-spiro[1-benzofuran-2,4′-piperidin]-1′-yl)-3-[5-fluoro-2-(hydroxymethyl)phenoxy]propan-2-ol

A mixture of 5-chloro-3H-spiro[1-benzofuran-2,4′-piperidine] (98 mg) and {4-fluoro-2-[(2S)-oxiran-2-ylmethoxy]phenylImethanol (87 mg) in dry ethanol (10 ml) was stirred at 80° C. for 18 h. Then the solvent was removed in vacuo affording 183 mg of the subtitled compound, which was used without further purification.

APCI-MS: m/z 422 (MH⁻).

### Step 3: (2-{[(2S)-3-(5-Chloro-1′H,3H-spiro[1-benzofuran-2,4′-piperidin]-1′-yl)-2-hydroxypropyl]oxy}-4-fluorophenyl)methanesulfonic acid

Polymer-bound triphenylphosphine (3 mmol/g, 83 mg, 0.25 mmol) was stirred in dichloromethane (10 ml) for 30 min. (2S)-1-(5-chloro-l′H,3H-spiro[1-benzofuran-2,4′-piperidin]-1′-yl)-3-[5-fluoro-2-(hydroxymethyl)phenoxy]propan-2-ol (183 mg) was added, followed by tetrachloromethane (100 μl), and the mixture was stirred at room temperature for 18 h. An additional portion of tetrachloromethane (2 ml) and polymer-bound triphenylphosphine (3 mmol/g, 166 mg, 0.5 mmol) was added, and stirring was continued for 7 h., after which the insoluble material was removed by filtration, and the solvent removed in vacuo affording a brownish oil, which was redissolved in ethanol (2 ml). The solution was added to a suspension of sodium sulphite (1.0 g) in water (1 ml). The mixture was stirred for 18 h at 80° C. The inorganic material was removed by filtration, and the product purified by HPLC to afford 5 mg of the title compound.

¹H-NMR (CD₃OD, 400 MHz): δ 7.37 (dd, J=8.2, 6.8 Hz, 1H), 7.20 (s, 1H), 7.10 (dd, J=8.5, 2.1 Hz, 1H), 6.81 (dd, J =10.9, 2.4 Hz, 1H), 6.74 (d, J=8.3 Hz, 1H), 6.70 (td, j=8.4 2.3Hz, 1H), 4.39 (br.s, 1H), 4.15 (s, 1H), 4.12 (d, J =4.2 Hz, 1H), 4.09 (d, J=5.7 Hz, 1H), 4.06 (s, 1H), 3.76-3.37 (m, 6H), 3.14 (s, 2H), 2.18 (s, 4H).

APCI-MS: m/z 486 (MH⁺).

### Example 8

### Inflammatory Cell Influx Experiment in LPS-Challenged Rats

The effect of a CCR1 receptor antagonist N-(2-{[(2S)-3-(5-chloro-1′H,3H-spiro[1-benzofuran-2,4′-piperidin]-1′-yl)-2-hydroxypropyl]oxy}-4-hydroxyphenyl)acetamide, (referred to here as Compound B) and budesonide, and their combination, on inflammatory cell influx was assayed by monitoring the effect on total cell number in broncholalveolar lavage (BAL) fluid of rats challenged intra-tracheally (i.t.) with Lipopolyaccharide (LPS) [N=10 rats per treatment group].

Methodology

LPS instillation: Rats were anaesthetized with Isofluran and put in a supine position, head up, on a board tilted at 30°. LPS (Lipopolysaccharide B. E.coli 026:B6) (2.5 μg/ml) dissolved in saline (0.9% NaCl), or saline alone (negative control) in a volume of 200 μl was administered i.t. using a modified metal cannula. Rats remained in this position until regaining consciousness.

Preparation of solutions: Homogenised budesonide was dissolved in vehicle containing the following ingredients (mg/ml): sodium chloride (8.5), EDTA (0.1), citric acid dried (0.15), sodium citrate (0.5), polysorbat 80 (0.2) in Milli-Q water. Budesonide was homogenised in Polysorbat 80 and water by using dispersing tool “Ultra turrax”. The homogenised budesonide was then added to the vehicle at a concentration of 2.0 μg budesonide/ml. Compound B was dissolved in Vehicle solution to a final concentration of 0.01 or 10 μg/ml compound B.

Budesonide/compound B mixed formulations were made by dissolving compound B in the budesonide suspensions, giving a final concentration of 0.01 or 10 μg compound B/ml and 2 μg budesonide/ml.

Treatments: Animals were intratracheally instilled with solutions (1 ml/kg) of budesonide/compound B (2.0/0.1-10 μg/kg), or of budesonide (2.0 μg/kg) alone, or compound B (0.01 or 10 μg/kg) alone, or with Vehicle (negative and positive control animals). The treatments were carried out under light anaesthesia (Isofluran) to secure that the solution reached the lungs. The drugs were administrated 30 min before the LPS instillation.

Termination: 4 hours after the LPS challenge, rats were intraperitoneally injected with the mixture (2 ml) of pentobarbital (60 mg/ml, Apoteksbolaget, Sweden) and PBS (1:1) for 1-2 min.

Bronchoalveolar lavage: After termination, BAL was performed twice with PBS. The BAL fluid was centrifuged and the cell pellet was resuspended in PBS. The total numbers of BAL cells were counted in a SYSMEX cell counter.

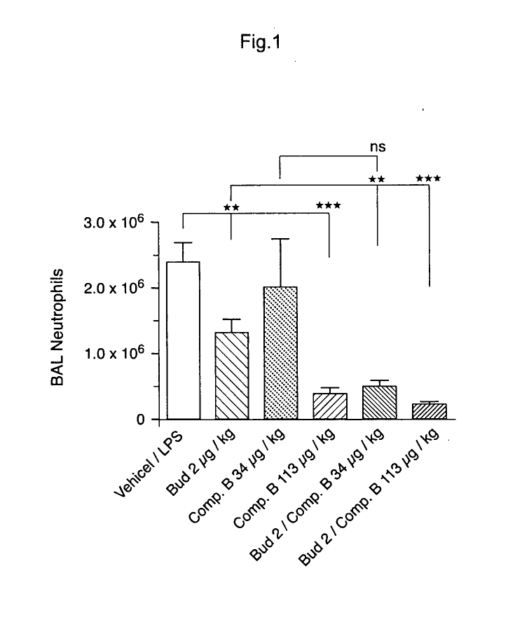

The results of the experiment are shown in FIG. 1. In FIG. 1 “vehicle/saline” rats represents the negative control rats treated with vehicle and challenged with saline. “vehicle/LPS” animals represent the positive control rats treated with vehicle and challenged with LPS. The remaining five groups were all treated with the specified drugs and challenged with LPS.

1. A pharmaceutical product comprising, in combination,
(a) a first active ingredient, which is a compound of general formula (I): 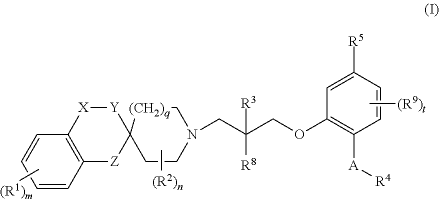
wherein:
m is 0, 1 or 2;
R¹ is halogen, cyano or C₁-₆haloalkyl;
X, Y and Z are independently a bond, —O—, —NH—, CH₂— or —C(O)—, provided that only one of X, Y and Z is a bond, and provided that X and Y are not simultaneously —O— or —C(O)—;
n is 0, 1 or 2;
R² is ═O or C₁-₆alkyl;
q is 0 or 1;
R³ is hydrogen, hydroxyl or NH₂;
R⁸ is hydrogen or C₁-₆alkyl;
A is a bond or C₁-₃alkyl;
R⁴ is hydrogen, hydroxyl, oxo, NHC(O)R¹⁰, C(O)NR¹¹R¹², COOR¹³ or SO₃R¹³;
R⁵ is hydrogen, halogen, hydroxyl or C₁-₆alkoxy, optionally substituted by one or more substituent independently selected from halogen, cyano, hydroxyl and carboxyl;
t is 0, 1 or 2;
R⁹ is halogen, cyano, C₁-₃alkoxy or C₁-₃haloalkyl;
R¹⁰ is hydrogen, C₁-₃alkyl, NR¹¹R¹² or OR¹³;
R¹¹ and R¹² are independently selected from hydrogen, C₁-₆alkyl and C₃-₇cycloalkyl, or R¹¹ and
R¹² together with the nitrogen atom to which they are attached form a 4 to 7-membered heterocyclic ring, which may optionally be substituted by one or more hydroxyl groups; and
R¹³ is hydrogen or C₁-₃alkyl,
or a pharmaceutically acceptable salt thereof; and
(b) a second active ingredient, which is a glucocorticoid receptor agonist; and optionally
(c) a third active ingredient, which is a β₂-agonist, provided the agonist is not selected from a N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide or a salt thereof,
a N-[2-(Diethylamino)ethyl]-N-(2-{[2-(4-hydroxy-2-oxo-2,3-dihydro-1,3-benzothiazol-7-yl)ethyl]amino}ethyl)-3-[2(3-chlorophenyl)ethoxy]propanamide or a salt thereof, or
a 7-[(1R)-2-({2-[(3-{[2-(2-Chlorophenyl)ethyl]amino}propyl)-thio]ethyl}amino)-1-hydroxyethyl]-4-hydroxy-1,3-benzothiazol-2(3H)-one or a salt thereof;.

2. The pharmaceutical product according to claim 1, comprising, in combination,
(a) a first active ingredient, which is a compound of general formula (I) wherein:
m is 1;
R¹ is halogen; X, Y and Z are independently a bond, —O— or CH₂—, provided that only one of X, Y and Z is a bond; n is 0; q is 1; R³ is hydroxyl; R⁸ is hydrogen; A is a bond; R⁴ is C(O)NR¹¹R¹²;
R⁵ is C₁-₆alkoxy, optionally substituted by one or more substituent independently selected from hydroxyl and carboxyl; t is 1; R⁹ is halogen; R¹¹ and R¹² are independently selected from hydrogen and C₁-₆alkyl; or a pharmaceutically acceptable salt thereof; and
(b) a second active ingredient, which is a glucocorticoid receptor agonist; and optionally
(c) a third active ingredient, which is a β₂-agonist, provided the agonist is not selected from a N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide or a salt thereof,
a N42-(Diethylamino)ethyl]-N-(2-{[2-(4-hydroxy-2-oxo-2,3-dihydro-1,3-benzothiazol-7-yl)ethyl]amino}ethyl)-3-[2-(3-chlorophenyl)ethoxy]propanamide or a salt thereof, or a 7-[(1R)-2-({2-[(3-{[2-(2-Chlorophenyl)ethyl]amino}propyl)-thio]ethyl}amino)-1-hydroxyethyl]-4-hydroxy-1,3-benzothiazol-2(3H)-one or a salt thereof.

3. The pharmaceutical product according to claim 1, wherein the compound of formula (I) is selected from: N-(2-{[(2S)-3-(5-chloro-1′H,3H-spiro[1-benzofuran-2,4′-peperidin]-1′-yl)-2-hydroxypropyl]oxy}-4-hydroxyphenyl)acetamide;
N-(2-{[(2S)-3-(5-chloro-1′H-spiro[1,3-benzodioxole-2,4′-piperidin]-1′-yl)-2-hydroxypropyl]oxy}-4-hydroxyphenyl)acetamide trifluoroacetate (salt);
2-{[(2S)-3-(5-chloro-1′H,3H-spiro[1-benzofuran-2,4′-piperidin]-1′-yl)2hydroxypropyl]oxy}-4-hydroxy-N-methylbenzamide trifluoroacetate (salt);
2-{[(2S)-3-(5-chloro-1′H,3H-spiro [1-benzofuran-2,4′-piperidin]-1′-yl)-2-hydroxypropyl]oxy}-4-hydroxybenzoic acid trifluoroacetate (salt);
N-(2-{[(2S)-3-(5-chloro-1′H,3H-spiro[2-benzofuran-1,4′-piperidin]-1′-yl)-2-hydroxypropyl]oxy}-4-hydroxyphenyl)acetamide trifluoroacetate (salt);
2-{[(2S)-3-(5-chloro-1′H,3H-spiro[2-benzofuran-1,4′-piperidin]-1′-yl)-2-hydroxypropyl]oxy}-4-hydroxy-N-methylbenzamide;
N-(2-{[(2S)-3-(5-fluoro-1′H,3H-spiro[1-benzofuran-2,4′-piperidin]-1′-yl)-2-hydroxypropyl]oxy}-4-hydroxyphenyl)acetamide;
2-{[(2S)-3-(5-fluoro-1′H,3H-spiro[1-benzofuran-2,4′-piperidin]-1′-yl)-2-hydroxypropyl]oxy}-4-hydroxy-N-methylbenzamide;
N-[2-({(2S)-3-[(2R)-5-chloro-1′H,3H-spiro[1-benzofuran-2,3′-pyrrolidin]-1′-yl]-2-hydroxypropyl}oxy)-4-hydroxyphenyl]acetamide;
N-(2-{[(2S)-3-(5-chloro-1′H,3H-spiro[1-benzofuran-2,4′-piperidin]-1′-yl)-2-hydroxypropyl]oxy}-4-hydroxyphenyl)urea trifluoroacetate (salt);
4-fluoro-2-{[(2S)-3-(5-fluoro-1′H,3H-spino[1-benzofuran-2,4′-piperidin]-1′-yl)-2-hydroxypropyl]oxy}benzoic acid hydrochloride;
N-(2-{[(2S)-3-(5-chloro-1′H,3H-spiro[1-benzofuran-2,4′-piperidin]-1′-yl)-2-hydroxypropyl]oxy}-4-fluorophenyl)urea trifluoroacetate (salt);
N-(2-{[(2S)-2-amino-3-(5-fluoro-1′H,3H-spiro[1-benzofuran-2,4′-piperidin]-1′-yl)propyl]oxy}-4-hydroxyphenyl)acetamide bis(trifluoroacetate) (salt);
Benzaldehyde, 2-[(2S)-3-(5-chlorospiro[benzofuran-2(3H),4′-piperidin]-1′piperidin]-1′-yl)-2-hydroxypropoxy]- ;
Spiro[benzofuran-2(3H),4′-piperidine]-1′-ethanol, 5-chloro-α-[[2-(2-hydroxyethyl)phenoxy]methyl]-, (αS)-;
Spiro[benzofuran-2(3H),4′-piperidine]-1′-ethanol, 5-chloro-α-[[2-(hydroxymethyl)phenoxy]methyl]-, (αS)-;
N-(2-[(2S)-3-(5-chloro-1′H,3H-spiro[1-benzofuran-2,4′-piperidin]-1′-yl)-2-hydroxypropyl]oxy}-5-chloro-4-hydroxyphenyl)acetamide;
2-Chloro-5-[(25)-3-(5-chloro-1′H,3H-spiro[1-benzofuran-2,4′-piperidin]-1′-yl)-2-hydroxypropyl]oxy}-(4-{acetylamino}phenoxy)acetic acid;
5-{[(2S)-3-(5-Chloro-1′H,3H-spiro[1-benzofuran-2,4′-piperidin]-1′-yl)-2-hydroxypropyl]oxy}-(4-{acetylamino}phenoxy)acetic acid;
{2-Chloro-5-[(2S)-3-(5-chloro-1′H,3H-spiro[1-benzofuran-2,4′-piperidin]-1′-yl)-2-hydroxypropyl]oxy}-4-[(methylamino)carbonyl]phenoxy}acetic acid;
2-{2-Chloro-5-[(2S)-3-(5-chloro-1′H,3H-spiro [1-benzofuran-2,4′-piperidin]-1′-yl)-2-hydroxypropyl]oxy}-4-[(methylamino)carbonyl]phenoxyl-2-methylpropanoic acid;
(2-Chloro-5-{[(2S)-3-(5-chloro-1′H,3H-spiro[1-benzofuran-2,4′-piperidin]-1′-yl)-2-hydroxypropyl]oxy}-4-{[(3S)-3-hydroxypyrrolidin-1-yl]carbonyl}phenoxy)acetic acid;
5-Chloro-2-{[(2S)-3-(5-chloro-1′H,3H-spiro[1-benzofuran-2,4′-piperidin]-1′-yl)-2- hydroxypropyl]oxy}-4-(cyanomethoxy)benzoic acid tifluoroacetate (salt);
2-{[(2S)-3-(5-chloro-1′H,3H-spiro[1-benzofuran-2,4′-piperidin]-1′-yl)-2-hydroxypropyl]oxy}-5-chloro-4-(2,2-difluoroethoxy)benzoic acid trifluoroacetate (salt);
5-Chloro-2-{[(2S)-3-(5-chloro-1′H,3H-spiro[1-benzofuran-2,4′-piperidin]-1′-yl)-2-hydroxypropyl]oxy}-4-(3,3,3-trifluoropropoxy)benzoic acid trifluoroacetate (salt);
N-(2-{3-[5-chloro-1′H,3H-spiro[1-benzofuran-2,4′-piperidin]-1′-yl]propoxy}phenyl)acetamide trifluoroacetate (salt);
Methyl 3-(2-{[(2S)-3-(5-chloro-1′H3H-spiro[1-benzofuran-2,4′-piperidin]-1′-yl)-2-hydroxypropyl]oxy}-4-fluorophenyl)propanoic acid trifluo acetic acid salt;
N-(2-{[(2S)-3-({spiro[indole-2-4′-piperidin]-3(1H)-one}-1′-yl)-2hydroxypropyl]oxy}-4-hydroxyphenypacetamide; and
(2-{[(2S)-3-(5-Chloro-1′H,3H-spiro [1-benzofuran-2,4′-piperidin]-1′-yl)-2-hydroxypropyl]oxy}-4-fluorophenyl)methanesulfonic acid
or a pharmaceutically acceptable salt, solvate or solvated salt thereof.

4. A pharmaceutical product comprising, in combination,
(a) a first active ingredient, which is 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid or pharmaceutically acceptable salts, solvate or solvated salt thereof; and
(b) a second active ingredient, which is a glucocorticoid receptor agonist; and optionally
(c) a third active ingredient, which is a β₂-agonist, provided the agonist not selected from
a N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide or a salt thereof,
a N-[2-(Diethylamino)ethyll-N-(2-{[2-(4-hydroxy-2-oxo-2,3-dihydro-1,3-benzothiazol-7-yl)ethyl]amino}ethyl)-3-[2-(3-chlorophenyl)ethoxy]propanamide or a salt thereof, or
a 7-[(1R)-2-({2-[(3-{[2-(2-Chlorophenyl)ethyl]amino}propyl)-thio]ethyl}amino)-1-hydroxyethyl]-4-hydroxy-1,3-benzothiazol-2(3H)-one or a salt thereof

5. The pharmaceutical product according to claim 1, wherein the glucocorticoid receptor agonist is budesonide.

6. The pharmaceutical product according to claim 1, wherein the β₂-agonist is selected from any of formoterol, indacaterol or a N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide or a salt thereof,
a N-[2-(Diethylamino)ethyl]-N-(2-{[2-(4-hydroxy-2-oxo-2,3-dihydro-1,3-benzothiazol-7-yl)ethyl]amino}ethyl)-3-[2-(3-chlorophenyl)ethoxy]propanamide or a salt thereof, or
a 7-[(1R)-2-({2-[(3-{[2-(2amino}propyl)-thio]ethyl}amino)-1-hydroxyethyl]-4-hydroxy-1,3-benzothiazol-2(3H)-one or a salt thereof.

7. The pharmaceutical product according to claim 1, which is in a form suitable for administration by inhalation.

8. The pharmaceutical product according to claim 1 for use in therapy.

9. (canceled)

10. (canceled)

11. A method of treating airway diseases which comprises simultaneously, sequentially or separately administering:
(a) a (therapeutically effective) dose of a first active ingredient, which is a compound of formula (I) or a pharmaceutically acceptable salt thereof as defined in claim 1;
and
((b) a (therapeutically effective) dose of a second active ingredient, which is a glucocorticoid receptor agonist or a pharmaceutically acceptable salt thereof; and optionally, (c) a (therapeutically effective) dose of a third active ingredient, which is a β₂ agonist, provided the agonist not selected from
a N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide or a salt thereof,
a N-[2-(Diethylamino)ethyl]-N-(2-{[2-(4-hydroxy-2-oxo-2,3-dihydro-1,3-benzothiazol-7-yl)ethyl]amino}ethyl)-3-[2-(3-chlorophenyl)ethoxy]propanamide or a salt thereof, or a 7-[(1R)-2-({2-[(3-{[2-(2-Chlorophenyl)ethyl]amino}propyl)-thio]ethyl}amino)-1-hydroxyethyl]-4-hydroxy-1,3-benzothiazol-2(3H)-one or a salt thereof;
to a patient in need thereof.

12. The method according to claim 11, wherein (a) the first active ingredient is 2-{2-Chloro-5-[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid or a pharmaceutically acceptable salt thereof, and
(b) a second active ingredient is budesonide; and optionally,
(c) a third active ingredient is selected from any of formoterol, indacaterol or
a N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide or a salt thereof,
a N-[2-(Diethylamino)ethyl]-N-(2-{[2-(4-hydroxy-2-oxo-2,3-dihydro-1,3-benzothiazol-7-yl)ethyl]amino}ethyl)-3-[2-(3-chlorophenyl)ethoxy]propanamide or a salt thereof, or
a 7-[(1R)-2-({2-[(3-{[2-(2-Chlorophenyl)ethyl]amino}propyl)-thio]ethyl}amino)-1-hydroxyethyl]-4-hydroxy-1,3-benzothiazol-2(3H)-one or a salt thereof.

13. A pharmaceutical composition comprising, in admixture, a) a (therapeutically effective) dose of a first active ingredient, which is a compound of formula (I) or a pharmaceutically acceptable salt thereof as defined in claim 1; and
((b) a (therapeutically effective) dose of a second active ingredient, which is a glucocorticoid receptor agonist or a pharmaceutically acceptable salt; and optionally,
(c) a (therapeutically effective) dose of a third active ingredient, which is a β₂ agonist, thereof provided the agonist not selected from
a N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide or a salt thereof,
a N-[2-(Diethylamino)ethyl]-N-(2-{[2-(4-hydroxy-2-oxo-2,3-dihydro-1,3-benzothiazol-7-yl)ethyl]amino}ethyl)-3-[2-(3-chlorophenyl)ethoxy]propanamide or a salt thereof, or
a 7-[(1R)-2-({2-[(3-{[2-(2-Chlorophenyl)ethyl]amino}propyl)-thio]ethyl}amino)-1-hydroxyethyl]-4-hydroxy-1,3-benzothiazol-2(3H)-one or a salt thereof;
with pharmaceutically acceptable adjuvants, diluents and/or carriers.

14. Compounds selected from
5-Chloro-2-{[(2S)-3-(5-chloro-1′H,3H-spiro[1-benzofuran-2,4′-piperidin]-1′-yl)-2-hydroxypropyl]oxy}-4-(cyanomethoxy)benzoic acid;
2-{[(2S)-3-(5-chloro-1′H,3H-spiro[1-benzofuran-2,4′-piperidin]-1′-yl)-2-hydroxypropyl]oxy}-5- chloro-4-(2,2-difluoroethoxy)benzoic acid;
5-Chloro-2-{[(2S)-3-(5-chloro-1′H,3H-spiro[1-benzofuran-2,4′-piperidin]-1′-yl)-2-hydroxypropyl]oxy}-4-(3,3,3-trifluoropropoxy)benzoic acid;
N-(2-{3-[5-chloro-1′H,3H-spiro[1-benzofuran-2,4′-piperidin]-1′-yl]propoxy}phenyl)acetamide;
Methyl 3-(2-{[(2S)-3-(5-chloro-1′H3H-spiro[1-benzofuran-2,4′-piperidin]-1′-yl)-2-hydroxypropyl]oxy}-4-fluorophenyl)propanoic acid; and
N-(2-{[(2S)-3-({spiro[indole-2-4′-piperidin]-3(1H)-one}-1′-yl)-2-hydroxypropyl]oxy}-4-hydroxyphenypacetamide,
or pharmaceutically acceptable salts, solvate or solvated salt thereof.

15. The method according to claim 11, wherein the airway disease is chronic obstructive pulmonary disease or asthma.
((b) a (therapeutically effective) dose of a second active ingredient, which is a glucocorticoid receptor agonist or a pharmaceutically acceptable salt; and optionally,
(c) a (therapeutically effective) dose of a third active ingredient, which is a β₂ agonist, thereof provided the agonist not selected from
a N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide or a salt thereof,
a N-[2-(Diethylamino)ethyl]-N-(2-{[2-(4-hydroxy-2-oxo-2,3-dihydro-1,3-benzothiazol-7-yl)ethyl]amino}ethyl)-3-[2-(3-chlorophenyl)ethoxy]propanamide or a salt thereof, or
a 7-[(1R)-2-({2-[(3-{[2-(2-Chlorophenyl)ethyl]amino}propyl)-thio]ethyl}amino)-1-hydroxyethyl]-4-hydroxy-1,3-benzothiazol-2(3H)-one or a salt thereof;
with pharmaceutically acceptable adjuvants, diluents and/or carriers.

14. Compounds selected from
5-Chloro-2-{[(2S)-3-(5-chloro-1′H,3H-spiro[1-benzofuran-2,4′-piperidin]-1′-yl)-2-hydroxypropyl]oxy}-4-(cyanomethoxy)benzoic acid;
2-{[(2S)-3-(5-chloro-1′H,3H-spiro[1-benzofuran-2,4′-piperidin]-1′-yl)-2-hydroxypropyl]oxy}-5-chloro-4-(2,2-difluoroethoxy)benzoic acid;
5-Chloro-2-{[(2S)-3-(5-chloro-1′H,3H-spiro[1-benzofuran-2,4′-piperidin]-1′-yl)-2-hydroxypropyl]oxy}-4-(3,3,3-trifluoropropoxy)benzoic acid;
N-(2-{3-[5-chloro-1′H,3H-spiro[1-benzofuran-2,4′-piperidin]-1′-yl]propoxy}phenyl)acetamide;
Methyl 3-(2-{[(2S)-3-(5-chloro-1′H3H-spiro[1-benzofuran-2,4′-piperidin]-1′-yl)-2-hydroxypropyl]oxy}-4-fluorophenyl)propanoic acid; and
N-(2-{[(2S)-3-({spiro[indole-2-4′-piperidin]-3(1H)-one}-1′-yl)-2-hydroxypropyl]oxy}-4-hydroxyphenyl)acetamide,
or pharmaceutically acceptable salts, solvate or solvated salt thereof.
